(19) United States
(12) Patent Application Publication
(10) Pub. No.: US 2015/0148632 A1
(43) Pub. Date: May 28, 2015

(54) Calorie Monitoring Sensor And Method For Cell Phones, Smart Watches, Occupancy Sensors, And Wearables

(71) Applicant: David Alan Benaron, Portola Valley, CA (US)

(72) Inventor: David Alan Benaron, Portola Valley, CA (US)

(21) Appl. No.: 14/552,468

(22) Filed: Nov. 24, 2014

Related U.S. Application Data
(60) Provisional application No. 61/908,926, filed on Nov. 26, 2013.
(60) Provisional application No. 61/970,667, filed on Mar. 26, 2014.
(60) Provisional application No. 61/989,140, filed on May 6, 2014.
(60) Provisional application No. 62/050,828, filed on Sep. 16, 2014.
(60) Provisional application No. 62/050,900, filed on Sep. 16, 2014.
(60) Provisional application No. 62/050,954, filed on Sep. 16, 2014.
(60) Provisional application No. 62/053,780, filed on Sep. 22, 2014.

(51) Int. Cl. A61B 5/083; A61B 5/00; A61B 5/1455

(52) U.S. Cl. CPC A61B 5/083; A61B 5/1455; A61B 5/0075; A61B 5/7225
USPC 600/322; 600/476

(57) ABSTRACT

An improved sensor (102) for calorie monitoring in mobile devices, wearables, security, illumination, photography, and other devices and systems uses an optional phosphor-coated broadband white LED (103) to produce broadband light (114), which is then transmitted along with any ambient light to target (125) such as the ear, face, or wrist of a living subject. Some of the scattered light returning from the target to detector (141) is passed through narrowband spectral filter set (155) to produce multiple detector regions, each sensitive to a different narrowband wavelength range, and the detected light is spectrally analyzed to determine a measure of calories, such as calories expended, calories ingested, calorie balance, or rate of calories expended, in part based on a noninvasive measure of respiration, such as respiratory rate, respiratory effort, respiratory depth, or respiratory variability. In one example, variations in concentration in components of the bloodstream over time, such as hemoglobin and water in the arteries, are determined based on the detected light, and the measure of respiration is then determined based on the variations in concentration over time. In the absence of the LED light, ambient light may be sufficient illumination for analysis. The same sensor can provide identifying features of type or status of a tissue target, such as heart rate or heart rate variability, hydration status, sleep state, or even occupancy counting. Calorie monitoring systems incorporating the sensor as well as methods are also disclosed.

### CROSS-REFERENCES TO RELATED APPLICATIONS

This application claims the benefit of, and priority to, U.S. Provisional Pat. Appn. No. 61/908,926, filed Nov. 26, 2013, U.S. Provisional Pat. Appn. No. 61/970,667, filed Mar. 26, 2014, and U.S. Provisional Pat. Appn. No. 61/989,140, filed May 6, 2014, U.S. Provisional Pat. Appn. No. 62/050,828, filed Sep. 16, 2014, U.S. Provisional Pat. Appn. No. 62/050,900, filed Sep. 16, 2014, U.S. Provisional Pat. Appn. No. 62/050,954, filed Sep. 16, 2014, U.S. Provisional Pat. Appn. No. 62/053,780, filed Sep. 22, 2014, U.S. Provisional Pat. Appn. No. 62/054,873, filed Sep. 24, 2014, the entire contents of each of which is incorporated herein in their entirety by this reference.

### FIELD OF THE INVENTION

The present invention relates generally to a calorie-sensing wearable device and method using light. More particularly, embodiments provide a filter-coated multi-element photodiode sensor for determining a calorie measurement in a living subject using variations in hemoglobin content of the bloodstream detected noninvasively using broadband light from ambient sunlight, ambient room light, or light from a solid-state broadband white LED. Enabling systems and methods for incorporating or practicing the improved calorie sensing are also disclosed

### BACKGROUND OF THE INVENTION

The traditional input for a consumer calorie-counting device is a set of accelerometers or gyros, or a GPS signal. These determine how much the body has physically moved. For example, pumping ones arms while walking allows an accelerometer to estimate energy spent. That is, they detect the motion of part of the body whenever a physical act such as exercise is performed, and calorie expenditure is then estimated based upon a correlation of a certain amount of physical movement (such as swinging the wrist while walking), with a typical level of calories expended. Another relationship could translate wrist movement to calories expended during running. More sophisticated schemes include GPS to determine distance walked or run, and elevation changes. In these cases, a certain distance travelled over a certain terrain in a certain way can be translated to an estimate of the calories expended. In both of these additional cases, these are physical actions that are detected as well.

What is common to these traditional physical approaches is that they are dependent on the physical action, but that they are insensitive to what the body is actually doing. For example, if we do not move, most of us still burn over 1,000 Calories in a day. This is the basal metabolic rate. However, a physical motion sensor will detect no activity. An algorithm using an accelerometer can correct for a minimum basal rate, but it is a correction . . . there is no physical motion sensed. Similarly, if we have a fever, we burn calories more quickly. However, an accelerometer or GPS based calorie sensor will not detect this. Further, weight lifting using a limb other than the one monitored by an accelerometer, or stationary cycling with a sensor one a non-moving wrist, fidgeting with one's feet, and other normal movements all affect calories expended, but are not detected by the traditional sensor.

Heart rate does change with exercise, and can be used to estimate calories, but heart rate is affected by blood flow not related to energy consumption, such as blood to the skin to cool the body on a hot day, or blood to the kidneys to increase urine flow. Thus, heart rate is not a direct measure of calories.

In the laboratory, the amount of oxygen used and calories expended, even at rest, can be measured in the breath, but such devices are typically cumbersome, requiring delivery of air with precisely known amounts of oxygen, sealed gas delivery systems, and gas analyzers unlikely to have appeal to the average mass consumer. Such a system would be difficult to use in the subject is ambulatory or exercising. In the end, however, it really should not matter if calories are expended on a stationary cycle or during the Tour de France, the user should be able to estimate calories spend. However there is a relationship between certain respiratory gasses and calories consumed, as is discussed later, that can enable the monitoring of calories using respirations.

Similarly, a user would like to know calories taken in via meals and fluids, without having to record the mass of the food or the volume of fluids ingested, and entering this data into a database. Accelerometers could record hand to mouth movement, but typically consumer devices miss this important task of eating and drinking. Knowing the calories ingested as well as the calories expended would allow for a determination of calorie balance.

Thus, conventional calorie monitoring mobile and wearable systems and methods suffer from one or more limitations noted above, in that they are not for mass consumer use, are difficult to use, reply on accelerometers or GPS and miss stationary expenditures, ignore calorie intake, and and/or they ignore or omit design considerations regarding optimizing calorie monitoring in living beings and tissues.

None of the above systems suggest or teach a method and system using light to estimate calorie expenditure, intake, balance, or rate in a manner that is a function of respiration, and therefore is responsive to physiology, including detecting basal caloric expenditure rate as well as changes not related to motion. Nor do the above systems teach estimation of calorie intake using light. More specifically, none of the above systems suggest or teach a method and system to monitor calories using arterial blood volume changes or other optical signatures associated with respirations. And none of the above systems work well for continuous monitoring of resting, ambulatory, or exercising subjects. Such a device for real-time sensing applications has not been taught, nor has such a tool been successfully commercialized.

### SUMMARY AND OBJECTS OF THE INVENTION

The present invention relies upon the discovery that certain features of physiology correlate with calories, and with the right measures, one can estimate calorie expenditures, intake, balance, and rate of expenditure. Similarly, such measures can be made to estimate caloric intake, allowing implementation more simply and inexpensively than has been achieved in a similar device and/or configuration using a conventional approaches. Such discovery led to development of a new sensor, allowing implementation more simply and inexpensively than has been achieved using conventional approaches.

A salient feature of the present invention is that mobile sensors and illuminators can detect metabolism (cytochrome or tissue oxygenation), respiratory load (respiratory rate, depth, effort, and variability), and that these measures can be correlated with actual calories used. Such that respiratory physiology and exercise caloric monitoring, can be beneficially enabled.

Another salient feature is that fat and water intake can be monitored, at the sensor level, at a low or reduced cost, enabling these caloric intake monitoring as well as caloric expenditure monitoring.

Another feature is that these determinations are useful over time, integrating the measures to yield a story over days, weeks, months, or years.

Another feature is that physiology, such as heart rate, respiratory rate, heart rate interval, arterial oxygenation, and tissue oxygenation can be extracted from these measures.

A final salient feature is recognition that such devices can be incorporated into many devices, including phones, watches, wristbands, pendants, traffic lights, street monitors, glasses, and the like. The device can be embedded in clothing (caps, belts, pants, sweats, shirts, suits), both for casual, work, and even professional use such as firefighters, police, pilots, and soldiers.

Accordingly, an object of the present invention is to provide a calorie sensor, including hardware and processing, to allow sensing and detection of type or state in mobile and imaging systems.

Another object is to provide a method for the stable detection calories expended, calories taken in, consumed or ingested, calorie balance, and rate of calories expended.

Another object is to provide these measures non-invasively.

Another object is to provide a method for the stable detection of the certain transient features of material sensed or imaged, such as to detect a rate of calories expended, heart rate, heart rate variability, respiratory rate, arterial oxygenation, and tissue oxygenation.

Another object is to provide a combination of a white or broadband LED, one or more spectral filters integrated with one or more optical sensors, and a processing layer into order to produce an integrated sensor/processor that provides a determination or result, such as calories expended or proximity of a hand, or even to measure other nearby bodies, such as to record the rates of calories expended by all persons in a business meeting in an image-only, non-contact manner.

Another object is to provide a sensor for embedding into nearly any mobile device, such as into a smartphone, personal wearable items (bracelet, pendant, watch, smart glasses, smart earbuds) and even into wearable clothing (shoe, shirt, or pants).

Another object is to provide an inexpensive spectral filter suitable for mass production.

Another object is to provide an inexpensive broadband solid-state light source of a configurable wavelength range.

The improved calorie sensor for mobile use as described has multiple advantages.

One advantage is that this improved sensor may now be safely deployed within cell phones, smart watches, or sports bracelets, wherein use of conventional sensors would have provided less information, been less reliable, been more costly, or been less functional. This includes in autos (for example, a sensor that analyzes your heart rate and alcohol content as an image in a non-contact manner before starting), or a military helmet (analyzes heart rate of all troops in your view, to identify subjects are risk for failure), or fitness clothing (analysis heart performance during a race), or medical monitors (alerts your physician when your heart is no longer working optimally).

Another advantage is that the improved sensors can enable new types of monitoring, from reliable non-contact sports monitoring to remote healthcare monitoring to business meeting monitoring in which the reactions and heart rates of all participants is known.

A final advantage is that the improved sensor, by virtue of its content-awareness or bio-awareness, can be incorporated into new devices and applications.

There is provided a calorie sensor for cell phones, health devices, and wearables. In one example, the system uses a phosphor-coated white LED and photodiodes with spectral filters, a processor, and software, to produce a system that reports on calories, such as calorie expenditure, calorie ingestion, calorie balance, and rate of calorie expenditure, when worn on the hand or finger, arm, ankle, face, ear, or other parts of the body, even in clothing. Systems incorporating this sensor for physiological monitoring, gesture enabling, and signature verification, and methods of use, are also described.

The breadth of uses and advantages of the present invention are best understood by example, and by a detailed explanation of the workings of a constructed apparatus, now in operation and tested in model systems and on human volunteers. These and other advantages of the invention will become apparent when viewed in light of the accompanying drawings, examples, and detailed description.

### BRIEF DESCRIPTION OF THE DRAWINGS

FIG. 1 is a schematic of an operating system using a cell phone and a small multispectral filter, constructed in accordance with the present invention.

FIG. 2A shows a fiber bundle multispectral filter.

FIG. 2B shows a photograph of a fiber bundle filter during testing.

FIG. 2C shows a sensor chip using spectral coatings on glass placed on a silicon detector chip, with collimating tubes and filter and shaping optics over each detector.

FIG. 2D shows a photograph of a sensor board built using coated spectral filters placed on silicon chip detectors constructed in accordance with the present invention.

FIG. 2E shows a schematic of a single-chip sensor chip.

FIG. 3A shows a broadband LED constructed from individual LEDs for use in the infrared.

FIG. 3B shows a photograph of a broadband infrared LED source array.

FIG. 4 shows the optical spectrum measured from a broadband infrared LED constructed in accordance with the present invention.

FIG. 5A shows a real-time, non-contact heart rate data stream, collected in this case 3-5 times a second from multispectral sensor in a cell phone constructed in accordance with the present invention.

FIG. 5B shows a real-time, non-contact heart rate data stream, collected from a multispectral sensor focusing on blood in the arterial supply as compared to a chest-lead medical EKG.

FIG. 6A shows data spectral data from a hand collected from a spectrally resolved sensor configured as a smart proximity detector to detect tissue, but not a book or a face.

FIG. 6B shows data spectral data from an arm with a sleeve covering the wrist collected from a spectrally resolved sensor configured as a smart proximity detector to detect tissue, but not a book or a face

FIG. 7 shows data from a wrist-based based sensor during exercise showing heart performance.

FIG. 8A shows a schematic side-view of a system incorporating the sensor into a loose-fit wristband.

FIG. 8B shows a schematic view of a system incorporating the sensor into a wristwatch.

FIG. 8C shows a system incorporating the sensor into a loose-fit non-contact pendant.

FIG. 8D shows a system incorporating the sensor into wearable glasses.

FIG. 8E shows a system incorporating the sensor into an energy-saving motion sensor for illumination control.

FIG. 8F shows a system incorporating the sensor into clothing.

FIG. 8G shows a system incorporating the sensor into an earphone earbud.

FIG. 9A shows a recessed non-contract sensor with the illumination and detection on the same chip.

FIG. 9B shows a non-contact recessed sensor where the white LED illuminator is separate from the detector.

FIG. 10A-B show respiratory rate detected using the arterial signal size. FIG. 10A shows loose fit oxy- and deoxy-hemoglobin data measured during exercise from a human subject over 100 seconds, with a filter with a time constant of 0.15 seconds, emphasizing the arterial pulse variations. FIG. 10B shows the same data with a 2 second time constant, emphasizing the arterial respiratory variations.

FIG. 11 shows a schematic flow chart of an approach incorporating the method of the present invention

FIG. 12A-B show data analyzed for oxygenation in accordance with the algorithm of the prior figure, and compartmentalized into venous and arterial compartments after both stabilization for skin changes, and differential analysis to emphasize changes over time. FIG. 12A shows calculations for changes in oxy- and deoxy-hemoglobin. FIG. 12B shows calculations resolved just to the arterial pulse compartment.

FIG. 13 shows using intervals to determine rate, in this case heart beat interval accuracy as determined by arterial compartment pulse and by EKG from data during exercise and movement, with a correlation coefficient of 0.94.

FIG. 14A-B show model data of how interval-based and counting-based rate estimation differ. FIG. 14A shows rate estimation in the presence of good data with no dropouts. FIG. 14B shows rate estimation in the presence of noise with some signal drop out.

FIG. 15 shows a plot of respiratory rate as measured and determined in accordance with the present invention on a human subject breathing at a controlled rate.

FIG. 16 shows cumulative calories expended as measured and calculated in accordance with the present invention on a human subject under study conditions.

FIG. 17 shows a multispectral signal detected using only ambient light.

FIG. 18 shows the selected components of a complex absorbance of hemoglobins, bilirubin, water, fat, and other substances.

FIG. 19A-B show data collected during movement of the sensor compared to the subject. FIG. 19A shows data during movement that obscures the heart rate effect by adding noise much larger than the signal. FIG. 19B shows data during movement but corrected for the movement using multispectral analysis.

FIG. 20A-B show data collected during movement of the subject but with a relatively stable sensor position. FIG. 20A shows data uncorrected for skin contact and blood volume changes that obscure the heartbeat. FIG. 20B shows the same data, corrected for probe movement, which reduces probe movement noise but does not correct for blood volume changes with body movement.

FIG. 21 shows raw data at six wavebands collected from a human subject during an exercise protocol.

### DEFINITIONS

For the purposes of this invention, the following definitions are provided:

Ambient Light: Light present in the environment. Ambient light is often broadband, that is available over a wide range of wavelengths to perform a detection or analysis, for example by solution of multiple simultaneous spectroscopic equations using a set of optical filters over a sensor. Sunlight is one type of ambient light. It appears white or off-white to the eye, and is also broadband (as defined below). Room light is another type of ambient light, and is of often broadband as well.

Loose-Fit: A device or sensor that, during movement, allows for a sensor to lift away from the body, without contact, but still allowing the sensor to continue monitoring. In contrast, most heart and respiratory monitors are tight-fit, requiring constant, snug contact with the skin or tissue of the subject being monitored. A tight fit forces light to travel into the skin, rather than reflecting back to the sensor, reduces blood movement in low-pressure venous compartments, and blocks ambient light from reaching the detector.

Compartment: A compartment is a location distinguished by temporal or physiological features that differentiate it from other locations. For example, the skin surface (which reflects and scatters light) can be one compartment. Muscle and tissue is another. The arterial bloodstream is a third example, and it differs in many respects (pressure, oxygenation, compliance) from the venous bloodstream, a fourth example of a compartment. Any region that can be differentiated based on such temporal or physiological characteristics can be a compartment for separation, localization, and computational analysis.

Occupancy: The presence, absence, or count of the living bodies in an area. An occupancy sensor could turn on a light if one or more human heartbeats are detected in a room (as opposed to or in addition to using motion to turn on the light), or an occupancy counter could turn up the air conditioning if 5 or more people's heartbeats are seen in a room. Processing spectral analysis of heartbeats using an image sensor with repeating groups of spectral sensors used to create “spectral pixel” groups, repeated as N×N over an image sensor would allow heartbeats to be spatially detected, temporally auto-correlated to establish identity, and counted.

Hydration Status: The overall water and fluid balance of an individual. In the simplest view, hydration reflects whether an individual has sufficient, insufficient, or excess body water. More complex analysis can look at which body compartments have water (such as intravascular fluids, extracellular fluids such as tissue edema, intracellular fluids).

Reduced-Power: Power consumption lowered as compared to similar sensors through the use of ambient light as a light source for some or all of sensor detection. Reduced power can be a relative term. For example, a sensor and LED system that does not require a lit broadband LED lamp at all times will use less power than an otherwise comparable design that always requires a lit broadband LED, allowing the ambient light system to operate on average at a lower power than the white LED dependent system. A reduction in power consumption by 20% would be considered reduced power.

Respiratory Rate: The rate at which breathing occurs. Breaths may be effective, ineffective (such as during obstruction), or even absent (such as in coma, or during certain types of sleep apnea). There are standard measures known to those skilled in the art, include breath volumes (tidal volumes), and the amount of air moved each minute (minute volume).

Content-blind: A gesture or event sensing approach that is dependent on a physical act or movement, but is insensitive to state, type, identity, or condition of the gesturer (subject) or object. For example, pressing a key on a keyboard is content-blind, as it does not matter if it is a pencil, a dead cat's paw, a monkey with a banana, or a user's finger that places physical pressure upon the keys or icon. In the view of typical smart phone keyboard, only the physical pressure of the object pressing the key (or for gesture sensitive devices, the movement of the touching object) is important, not the identity of the object doing the actuating.

Content-aware: In contrast to content-blind sensing, a sensing approach or system in which the sensor is able to intelligently detect and extract certain features about the person or object triggering the sensing event. For example, to analyze and detect that a hemoglobin-containing living hand or a chlorophyll-containing leaf appears in a photographic image are content-aware determinations. Content-awareness allows, for example, a proximity sensor to recognize that an object near a sensor is a living hand or finger, rather than a sleeve or a book, for specific gesture recognition with reduced error. This is not merely a pressure based or touch based system, such as a grip pressure sensor, but an actual spectral analysis to determine the type or state of the target, such as detection of hemoglobin, or changes in the hemoglobin concentration or volume in the bloodstream. Similarly, the color correction of a photograph can be improved if an image sensor is able to determine that a certain feature is human skin, or that another feature is sky, based on a spectral analysis of (or in additional to using traditional image processing of) the spectral information obtained by the sensor.

Bio-aware: A content-awareness that detects features of a living subject, such as the presence of hemoglobin, a heart rate, a body metabolism, a specific body composition, or recognition that an object near a sensor is a hand or finger for body-specific gesture recognition. A camera that color corrects pixels, or counts living objects present, based on the detection of hemoglobin in the one or more pixels, is bio-aware. This again is more than mere physical detection (such as a facial recognition algorithm using the shape of eyes and mouth) that would be fooled by a color photograph. A bio-aware method determines formal content such as chemical composition, not just physical appearance.

Filter: A device that restricts incoming light to of a specific type of light, such as by wavelength range, polarization, or other optical feature.

Spectral Filter: A filter that specifically restricts incoming light based on color or wavelength, usually restricting it to a predetermined set of colors or range or wavelengths, referred to herein as a waveband. For example, a narrowband interference coating that more or less allows only wavelengths from 550 to 560 nm to pass is a 10 nm bandwidth spectral filter for the waveband from 550 to 560 nm. Typical filters are Gaussian or have nearly vertical square sides, and each presents its own manufacturing advantages and challenges. For example, coating onto photodiodes is more difficult than coating on glass, as glass can survive much higher deposition temperatures without losing shape or function.

Sample or Target Sample: Material illuminated then detected by a sensor for bio-aware spectrally resolved analysis. A target sample may be an object, or can be living tissue.

Target Indicator: An optical characteristic specific to the target being measured.

Scattering: The redirection of light by a target sample. Most biological tissues scatter light, which is typically why we can see or detect them from light that scatters back from living tissues onto our retinae.

Light: Electromagnetic radiation from ultraviolet to infrared, namely with wavelengths between 10 nm and 100 microns, but especially those wavelengths between 200 nm and 2 microns, and more particularly those wavelengths between 400 and 1900 nm where chemical bands appear that allow unique identification.

Broadband Light: Light produced over a spectrally continuous and wide range of wavelengths (called the spectral width, spectral range, or bandwidth) sufficient to perform a detection or analysis, for example by solution of multiple simultaneous spectroscopic equations using a set of optical filters over a sensor. The broadband light could be ambient (such as from sunlight or room light), or it could be produced by sources such as a white LED integrated into the sensor. Spectral width is typically measured at some fraction of the peak intensity over the region of interest, such as full width half max (FWHM), full width quarter max (FWQM), or even full-width tenth max. For some purposes, a broadband range of at least 100 nm can at times be sufficient, while an exemplary sensor embodiment uses a white LED that produces light over 300 nm or more from 440 to 740 nm, with additional light is produced in a second broadband range of 880-1020 nm to provide additional analysis power, may be used. Ambient sunlight is broadband and covers a full UV, visible, and IR range from below 400 nm to above 2 microns.

Narrowband: The opposite of broadband is narrowband, and less than 50-100 nm in most cases. As a comparison, monochrome LEDs (non-laser, non-superluminescent) are often narrowband, with 20-70 nm widths, while narrowband filters used in the embodiments and examples herein can ideally be as narrow as 5 nm to 15 nm wide, with some more wide or more narrow.

Light Source: A source of illuminating photons. A light source can be external, such as sunlight.

LED: A light emitting diode.

White LED: A visible wavelength LED that appears white to the eye. For the purposes of this embodiment, the white LED is often a broadband white LED comprised of a blue LED and a broad-emitting blue-absorbing phosphor that emits over a wide range of visible wavelengths. Other phosphors can be substituted, including Lumigen or quantum dots.

Wearable: A sensor or device that can be worn on, in, or near the body, such as smart glasses, smart jewelry, or clothing with embedded sensors. The wearable can be an electronic device, like an earphone, an ocular implant or contact lens, a mouthpiece, tooth cover, prosthesis, or a monitoring band.

Motion: Movement, such as running during exercises.

Non-contact: A measurement in which the detector and/or the illuminator is not in contact with the tissue. This can be a short distance (such as a 2-10 mm spacing under a loose wristband), a medium distance (such as a security and movement detector on the ceiling of an office room, or an occupancy sensor or counter used to control illumination power), or a quite long distance (such as a glasses based sensor that overlays the heart and respiratory rate on people in your visual field even if both of you are in motion).

Loose Fit: A non-contact (or optionally non-contact) sensor or device configuration in which the data is collected without required contact with the tissue, such as a loose bracelet or a pendant.

Hemoglobin (or Heme): A pigmented molecule that carries oxygen in the blood. It is relevant to this invention that hemoglobin comes in many forms. In humans the primary forms are oxyhemoglobin (heme with oxygen) and deoxyhemoglobin (heme without oxygen). The reddish color of arterial blood comes from oxyhemoglobin being the main pigment (arterial hemoglobin is often over 96% oxyhemoglobin and under 4% deoxyhemoglobin), while the bluish color of venous blood is from the presence of large amounts of deoxyhemoglobin (venous hemoglobin is often around 30% deoxyhemoglobin with only 70% oxyhemoglobin).

Software: Software coded instructions for performing the method and algorithms taught herein are code stored on a non-transitory physical media, and are intended to direct a processor with memory, dedicated application-specific physical integrated circuit (ASIC), phone, fitness product, or other physical sensor systems to collect, analyze, and produce results from data collected from the sensors.

Measurement: A non-transient value determined over a period, or at one instant of time. A measurement is a stable form of information that can be stored in machine-readable hardware, such as a memory location, or can be used in mathematical equations or analysis.

### DETAILED DESCRIPTION

One embodiment of the device will now be described. This device has been built, and tested in the laboratory and on living subjects.

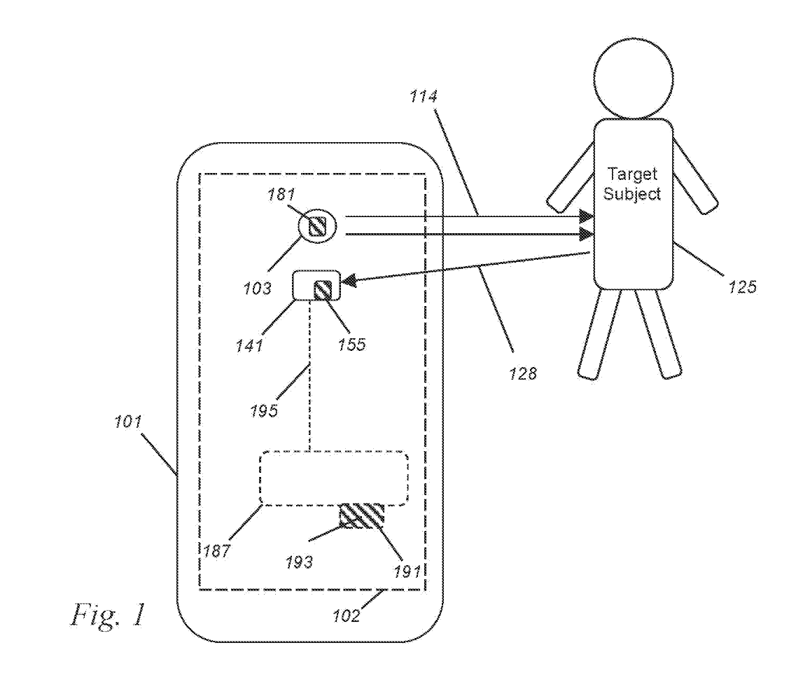

In the device source shown in FIG. 1, smart phone 101 has illuminator 103 and image camera detector 141. Illuminator 103, detector 141, and the processing, control circuitry, memory, and software together form sensor 102.

Illuminator 103 is a white LED. Broadband white light is emitted forward, in a beam as shown by light path vectors 114, with some light reaching (and optically coupled to) target site 125. Of note, target 125 is shown for illustrative purposes as a human subject, and is neither a part of the apparatus or system, nor is the human body or human subject claimed as patented material.

A portion of the light reaching target 125 is scattered and reflected, and returns as returning scattered and reflected light 128 into the smartphone camera image detector 141. Optionally, detector 141 could be a point detector, a linear array, or even one or more discrete detectors, provided that data representing filtered returning scattered light from the target sample is sensed and measured.

In this embodiment, detector 141 has added spectral filter 155. This filter allows only light of a certain color range onto certain pixel elements of detector 141. In this case, filter 155 may cover only a small region of the image sensor, so as not to interfere with image collection for other purposes, such as photographs. Filter 155 in this example has 7 narrowband filter ranges, each 5 nm FWHM wide, with center wavelengths at or near 525, 540, 555, 570, 585, 600, and 630 nm. Additional ranges may include filters with center wavelengths at or near 900, 920, 940, 960, and 980 nm for fat and water detection, and for these wavelengths in phones with white LED illumination, the 900-980 nm illumination must come from an infrared (IR) source in the phone's illumination or from ambient or other illumination sources). Sensor 102 measures less than 3 mm in width. Another range could be predetermined filters with center wavelengths at or near 445, 465, and 485 for the detection of bilirubin, the pigment of jaundice. Other filter sets could be selected for the detection of other compounds such as grain alcohol, sugar, abnormal hemoglobins, hematin (found in cells infected with malaria), and other biologically relevant pigmented molecules. Filter 155 is attached to detector 141 using optical epoxy.

The non-contact measurement can be enhanced using polarization filters, integrated into the emitter and at 90-degrees (cross-polarized) on the detector. This is because light that reflects off of the skin retains polarization, and can be blocked using a correctly positioned polarizer on the detector (in this case cross-polarized, but it may be a different angle in other situations). In contrast, light entering the tissue is depolarized during multiple scattering, and thus travels in greater percentages through the cross-polarizer on return, thus enhancing the light. In studies, we found that the apparent hemoglobin (a measure of travel through tissue) was up to 2-fold higher when crossed-polarizers were used. These are shown in FIG. 1 as a polarizer layer included as part of the construction of filter 155, and optional polarizer 181 over illuminator 103.

Next, some or all of the data from image detector 141, including the filtered pixels, is read and processed by embedded microcontroller processor 187 (such as those typically present to operate cell phones, and shown dashed as it is located internally as part of the cell phone main circuitry) based on machine-readable code 193 saved on physical media, such as ROM or flash disk memory 191, connected over electrical connection 195.

The machine readable code may optionally be system software saved as a machine-readable code embedded within a non-transitory physical memory ROM, or it could be an “app” (a downloadable code available for installation and/or purchase and then stored within a non-transitory physical memory), or it could be an “API” (an installed driver for a specific sensor, such as would be provided by a manufacturer with a given physical sensor set and using instructions stored on non-transitory computer readable media).

The precise design of software 172 will depend on the smartphone, watch, earbud, anklet, camera, or bracelet processor, but its function is to process the image and provide raw or processed results to the device or system For example, one result would be the photon counts for each of the filtered region, with each filter region covering multiple image pixels. Another result could be a processed result, in which least-squares fitting is performed against a spectral standard in order to determine the presence of hemoglobin in the image. Another result could be that the measurement is processed over time in order to produce a heart rate estimate. Each of these falls within the spirit of the invention if the returning light is processed for type, state, identify, or gesture, and if the broadband white LED source is used for illumination.

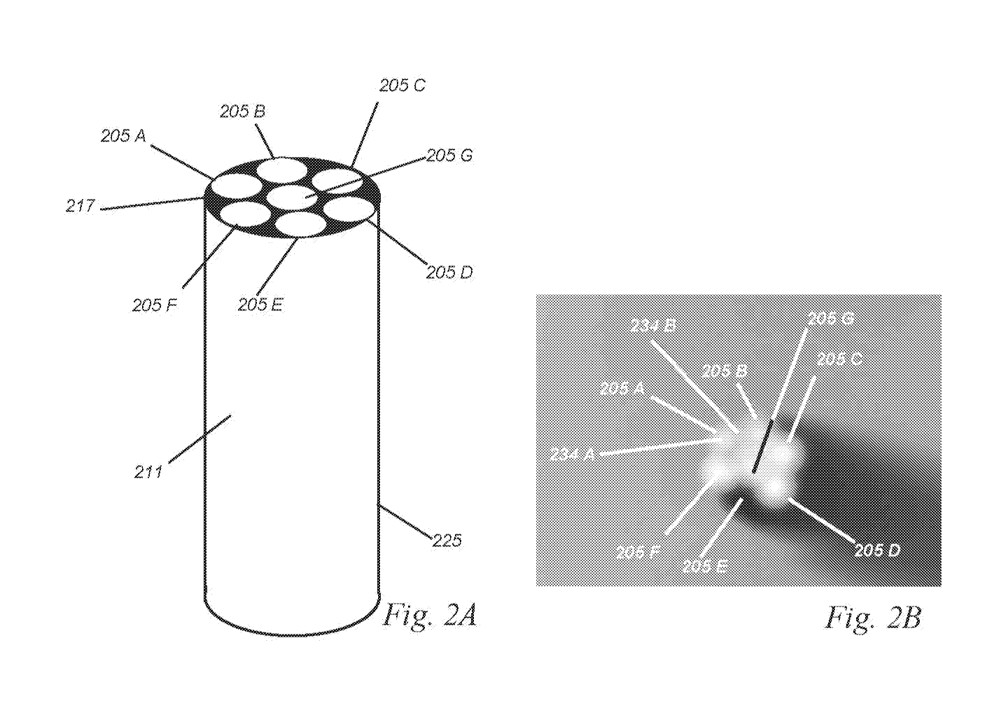

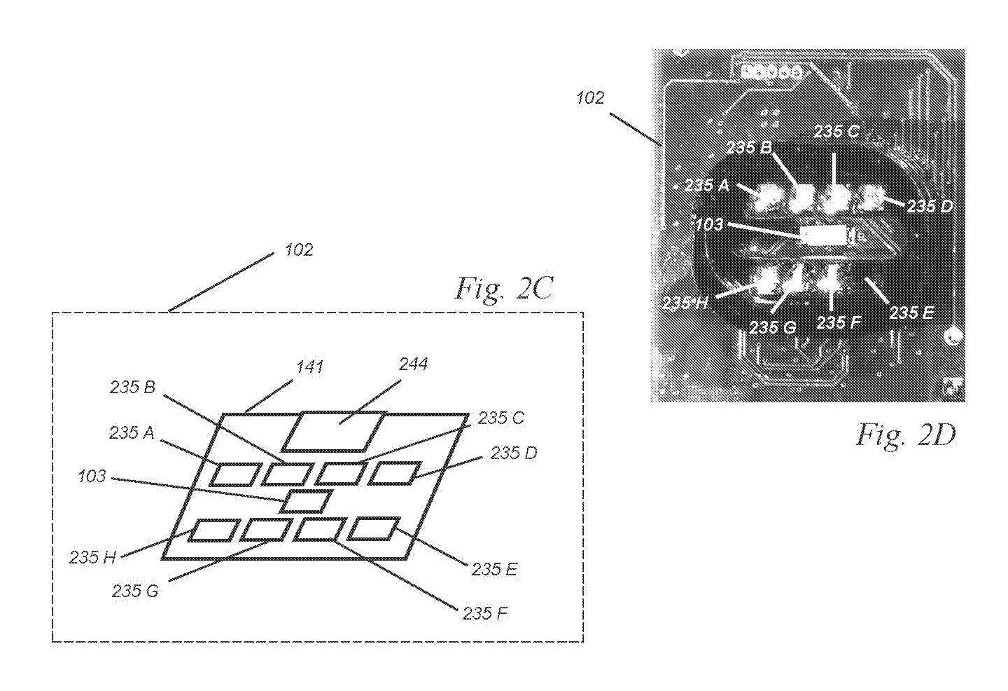

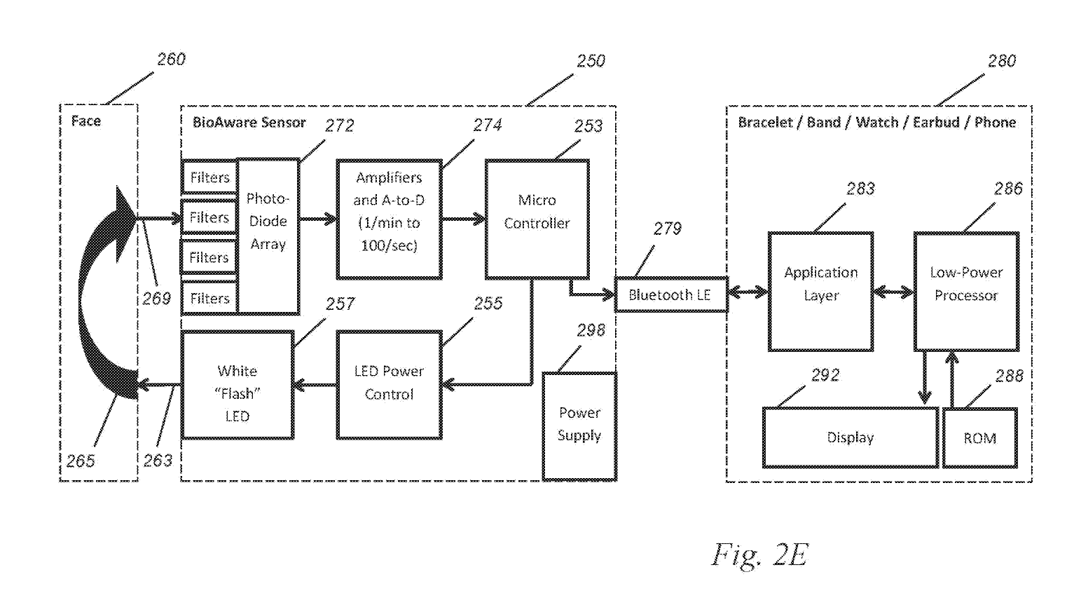

Spectral filter 155 of this preferred embodiment is now briefly described, as shown in FIG. 2. Here, filter 155 is shown as all of FIG. 2A and composed of 7 optical fibers 205A through 205G (one or more wavelengths described in Example 1 are omitted for clarity). The number of fibers can vary, even down to 1 but more typically 3 to 12, depending on application. Each of the fibers has a spectral filter coated onto the top end of each fiber, and the filter differs for each fiber 205A through 205G. In this example, the fibers are arranged in a circle of 6 outer fibers, with one central fiber. Alternatively, the fibers can be in various layouts, including different shaped patterns (square, linear row, star). In the construction of this custom filter, the fibers are first provided a filter coating, with each wavelength range run in a separate deposition chamber using pre-cut pre-polished (or cut) fibers, with thousands or more in each deposition chamber run. Then, one fiber of each wavelength is taken, prepared with epoxy on the side of the fiber, and placed into glass tube 211. Alternatively, tube 211 could be plastic, epoxy, resin, metal, or other material, provided it allows alignment and securing of the fibers. Once the black epoxy, shown as black epoxy 217, hardens, then distal end 225 can be polished.

A photograph of an actual 7-fiber system we constructed is shown in FIG. 2B, where all fibers (except fiber 205E) are illuminated. The image and localization improves with better polishing, spectral filter deposition, and other improvements to the fiber tip. This tip as shown in FIG. 2B can then be glued directly to detector 141 as shown in FIG. 1 as filter 155 on detector 141. The fibers are then attached (in this case, clear epoxy optical glue) to the face of the CCD for direct transfer of the transmitted photos to the image sensor detector.

Alternative constructions are optionally possible. For example, there may be more or fewer than 7 filter ranges, depending upon the intended application. Next, there may be more than 1 fiber for each wavelength range. For example, there may be 10 of each fiber, for a total of 700 fibers in the set. Then, after placement on the CCD, a calibration may need to be performed to assign each image sensor region to a pixel spectral range, allowing averaging and integration at several locations for each range.

Another alternative format for filter 155 as used in sensor 102 is shown as FIG. 2C. Here, the filter is comprised of a number of small filters assembled on one or more silicon detectors 141, shown as filters 235A through 235H, which are placed over the surface of detector(s) 141. Amplification and processing of the signals occurs in integrating amplifiers, and microcontroller processors, and instructions stored in non-transient machine-readable physical memory 244.

A photograph of such a device as constructed and tested is shown in FIG. 2D, where custom optical filters 235A-D and 235 F-H (Omega Optical, Brattleboro, Vt.) with collimating lenses can be seen on top of silicon photodiodes or phototransistors. Here elements are added above the silicon detectors to complete sensor chip 102, such as a collimating spacers, polarizers, and focusing lenses can be added, such as to reduce the angular bleed through of light into the spectral filters. The darker-appearing detector has only transparent region 235E in FIG. 2D, and no collimating lens, allowing unfocused and spectrally unfiltered white light to reach the detector).

A schematic of a sensor chip is shown in FIG. 2E. Here, sensor board 250 has microcontroller processor 253 (which can be an off-board controller) using LED power control 255 to power white LED 257 configured in flash mode. Light travels without tissue contact along light path 263 to a body part. As in FIG. 1, the human body and tissue are shown only to provide an understanding of the operation of the device, and the human body is not considered to be a claimed part of the present invention. Light scatters through the tissue along light path 265, and then leaves the tissue along backscattered and remitted light path 269, re-encountering sensor chip 250, and then enters spectral filter and photosensor detector array 272.

As described, the spectral filters can be separate elements, one filter element tuned by angle of entry across a range, or filters deposited directly on the detector substrate. In this case, interference filters were on separate glass substrates (custom 3×3 mm filters, Omega Optical, Brattleboro, Vt.) ranging from 5 to 40 nm FWHM, and were glued on each photodiode detector using optical quality UV set glue. A polarizer and lens were additionally added to the stack above each filter. The detector may be CMOS, a photodiode, a phototransistor, or any number of suitable optical detectors known in the art. In this example, the detectors are 8 photodiodes (Vishay temd7000 or larger). Alternatively, spectral filters could be replaced by a spectral grating that filters the light by spatially separating the wavelength into discrete wavebands over each physical region of light striking a detector.

Detector array 272 creates an output measureable amplified and digitized by amplifier and A-to-D converter 274. In this case, the detector outputs are captured and integrated by low noise CMOS or BiFET amplifiers (analog devices AD823A), and translated to 16-bit digital sample/hold A-to-D converter (Linear LTC1867L). High gain channels reach 66% saturation at 16 uW/cm². The measurement can be improved by use of MOSFET amplifiers, and also by using higher-gain phototransistors, or even avalanche photodiodes (though the required avalanche bias may increase the complexity of the chip and the cost of the sensor). Background estimation can be done by flashing the light at brief intervals. Each measurement filter channel is low pass filtered in two passive stages using a 1.2 ms time constant to control noise, and the light source itself is flashed on for 2 ms before a reading is taken. The system using less than 1 mm² of photodiode at each wavelength operates with 8-bit effective signal. By using a full 7.6 mm² from a 3×3 mm detector photodiode, 11 effective data bits can be obtained in this manner. For heart rate hemoglobin pulse signals, 8-14 bits is recommended. As shown in shown in FIG. 2E, signals leave board 250 and are transmitted over link 279 to a bracelet, band, watch, earbud, phone, or other device. Link 279 can be an I2C wire, or even a Bluetooth connection (such as Bluetooth Low Energy, or Bluetooth LE). Sensor 102 may encompass both board 250 as well as device 280, either as a stand-alone sensor or as an embedded system within another system, device, wearable, article of clothing, camera, sensor, or other device. Here, device 280 includes machine-readable non-transient machine code stored in stable, readable ROM 288, and executed in this case as app layer 283 running on processor 286. Display 292 may provide results, feedback, warnings, or upload confirmations to a user. It may even display messages from a concerned physician who is responding to the data collected by sensor 102.

Alternative formats are also possible for the broadband light source instead of using a single white LED.

Ambient sunlight is broadband and covers a full UV, visible, and IR range from below 400 nm to above 2 microns, while room light LEDs are increasingly found to be white broadband LEDs (as are illuminators in mobile phones). Alternative formats are also possible for an optional additional light source that may be needed to produce broadband light when the ambient light is dim.

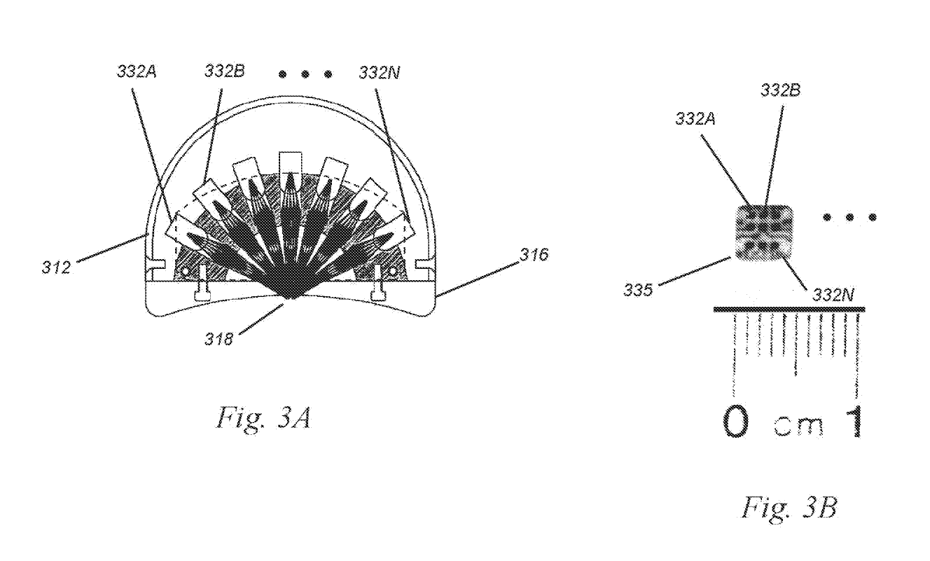

Another example is using a multiple narrowband LED source to create a broadband source, as shown in FIG. 3A. Such a combined LED may be required when measuring, for example, fat and water using the spectral peaks in the 700-1000 nm range, a region not supplied by most conventional white LEDs found in cell phones and other mobile devices. Here, frame 312 with bottom 316 and opening 318 holds multiple LEDs 332A to 332N. These multiple LEDs, which can include broadband LEDs such as a white LED, are inserted into frame 312. Light from the multiple LEDs is focused or concentrated out exit opening 318, to provide broadband light.

When manufactured, the light source can be significantly more compact, as shown in the photograph in FIG. 3B. Here, multiple LEDs 332A through 332N are surface mount LEDs on PC board 335.

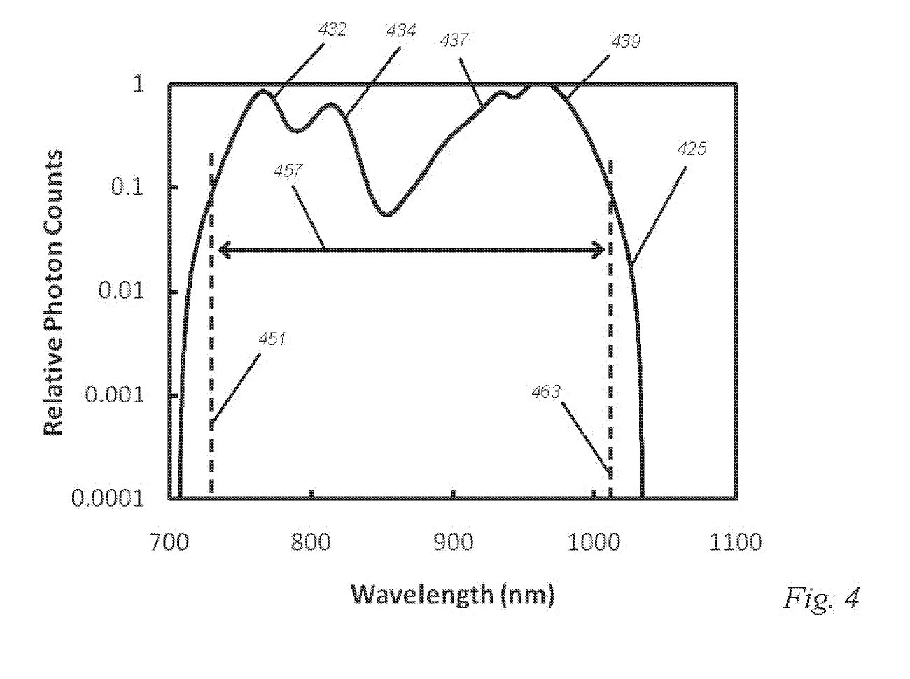

Light output from this multi-element light source is plotted in FIG. 4. Here, light emission is detected from about 700 nm to over 1000 nm, with light usable for over 300 nm of spectral width, from mark 451 to mark 463, with very little light by mark 425. Of note, the spectrum plotted shows peaks at peak 432, peak 434, peak 437, and peak 439, reflecting the peak contribution of certain LEDs used to build the light source. The width of the light output is shown as spectral width 457.

Operation of the device may now be described.

Smart phone 101 is turned on, and the spectral physiology app is selected by the user and started. For example, in an Android system, the app icon is located and touched, launching the app.

The app turns on phone white LED 103 and begins to collect data from camera detector 141. Data from detector 141 is accessed using software, in this case written in android language and compiled using the Android software development kit (SDK), available online (for example, at http://developer.android.com/sdk/index.html). Image data from detector 141 is available as RGB data (or as luminance and color, convertible to RGB using known equations). However, under spectral filter 155 the image from the lens is replaced by data from the fiber ends. An example of such data is shown in the image in FIG. 1, in this case collected using a USB plug in camera for a PC computer, dissembled and modified to have filter 155 attached and glued to the surface. The app software has already been calibrated to know which image pixels correspond to which filter, such as fiber center region 234A in FIG. 2B, and to ignore the overlap areas between fibers where two or more fibers overlap, such as fiber overlap region 234B in FIG. 2B. With many pixels of a detector covered by this filter, one may average the pixels to add statistical strength. What is produced by this combination is a table of the intensity at each of these wavelengths, which can then be analyzed in various ways.

This data may be collected on a spot basis for measurements without real-time change (such as water/fat composition), intermittently for values that change over minutes (such as cardiac performance), and nearly continuously (such as every 50 ms) for values such as heart rate, for which a continued change is key to extracting the value. These determinations are shown in more detail in the illustrative examples that follow.

### EXAMPLES

The breadth of uses of the present invention is best understood by examples, provided below. These examples are by no means intended to be inclusive of all uses and applications of the apparatus, merely to serve as case studies by which a person, skilled in the art, can better appreciate the methods of utilizing, and the scope of, such a device.

### Example 1

### Non-Contact Heart Rate Determination

In this example, illuminator 103 is a white LED embedded into a Samsung Galaxy S3 smartphone. Software app 172 is a custom software loaded into a machine-readable physical memory (4 Gb microSD card, San Disk) placed into the external SD card slot of the Galaxy phone, and installed using the Android operating system (Android 4.4, Google) on the phone. The app is launched using the Android touch interface. Multiple filters allowed multiple bands wavelength bands to be collected.

Upon launch, Software app 172 turns on illuminator 103, as well as displays a camera image from detector 141, which shows a hand placed into the image sensor view, but not necessarily in contact with the sensor. A pixel region corresponding to sensor intensity averaged over 100 pixels for each of these spectral ranges every 300 milliseconds is captured.

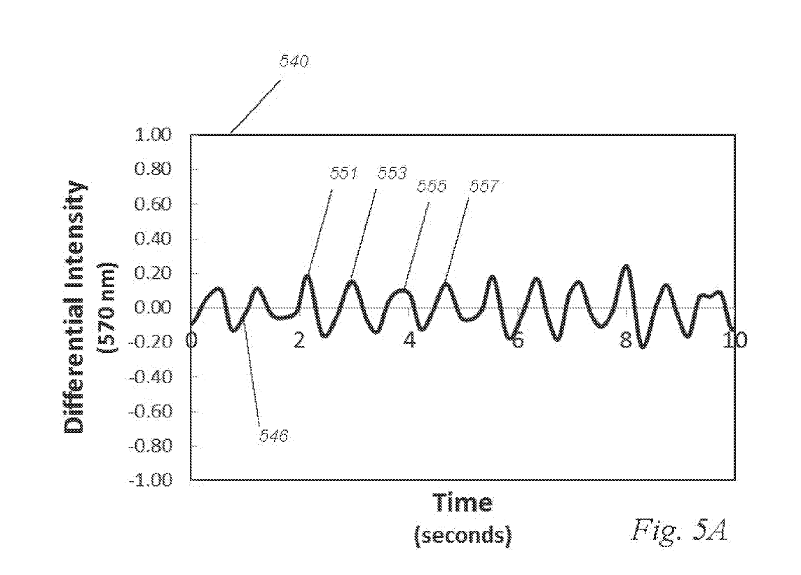

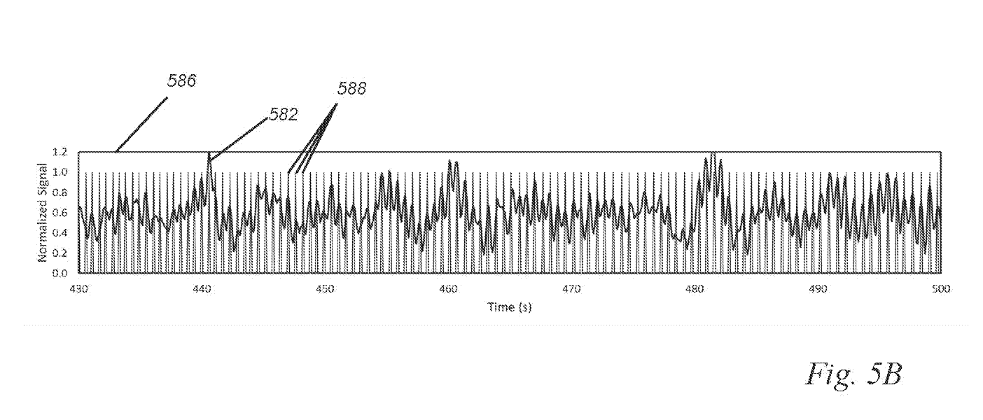

After capturing a spectral channel, the intensity is processed for change over time (a differential plot of intensity changes with respect to time). Here, the value is plotted versus time. The data are shown in FIG. 5A.

In FIG. 5, a time-varying output can be seen. In this case, the value of the output is determined as the normalized measurement from the 570 nm channel, minus a baseline change correction from a base-correction average of the measurement in the 460 and 630 nm channels. From this heart rate can be calculated simply by counting the peaks, using any of a number of methods familiar to those skilled in the art. One exemplary approach is to determine the beat-to-beat interval (i.e., the time between peaks). This allows for beats that are dropped to be detected as double-wide intervals which can be rejected, producing a more stable measurement in response to movement noise.

Alternatively, raw data, or interim determinations such as intensity changes over time, may optionally be displayed. Also, simply the changes in intensity at 570 nm (or other channels) may be plotted, as in a stable lighting environment the major change over intervals of seconds is the absorbance change caused by changes in hemoglobin.

For processing, a first differential (with respect to time) is determined, producing the varying measurement shown at plot 540 in FIG. 5A. Here, varying intensity 546 has peaks and troughs, which correspond to changes in hemoglobin volume with the pulsing of the heart. Peaks can be seen at 551, 553, 555, and 557. Each of these corresponds to one heartbeat. By determining a heart rate using the beat-to-beat intervals, and discarding the intervals with dropouts, a heart rate is determined; in this case, a heart rate of 72 beats/minute is measured and displayed.

Next, we constructed a research probe that allowed the sensor and broadband light source, of the types shown in FIG. 1 and constructed in accordance with the present invention, to be incorporated into a loose wristband system, with data collected at a multiple wavebands. How the data are processed to correct for physiology and motion are described in detail in later examples (for example, in Example 18 to Example 20).

Rather than use other indirect measures, such as other fitness monitors, we have compared the performance of this wristband to a chest electrode EKG, to test accuracy. Data were recorded from a human volunteer during an exercise protocol, as described in the previous example. This subject also wore an accelerometer, a pulse oximeter, and several other instruments that monitored multiple functions during the study.

The heart rate signal, as determined in accordance with the present invention in the previous example, in this case using 8 waveband multispectral data, is shown as plot line 582 in graph 586 of FIG. 5B. Also recorded at the same time, and plotted in FIG. 5B are the multiple, repetitive, narrow spikes of the QRS complex from a gold-standard chest lead EKG, shown as plot marks 588. The EKG records the electrical pulses from the heart with millisecond accuracy (when measured at 250 Hz with interpolation).

Comparing the signals visually at first, it can be seen by eye that there is a peak in the calculated heart rate signal with nearly every electrical signal, and very few such peaks visible where there is no EKG signal. This validates that the arterial signal has been extracted accurately, and that the timing of the signals is not invalidated by the EKG.

Instead of a visual assessment, another method of assessing the accuracy of these measures is to determine the interval between heartbeats, in milliseconds between beats or in effective heart rate at a given interval (e.g., an interval of 500 milliseconds corresponds to 120 beats/min), and compare these two measures. This beat-to-beat interval can be compared on a beat-to-beat basis, or averaged. In this following example, interval data were plotted as a running boxcar average over a moving 5-second window.

Several points are of note.

First, measurement of the heart rate occurred during hard exercise, and would have been noisy or unreadable if using just one wavelength. In order to perform this calculation, multiple wavelengths were used to correct for movement artifact, and pulsations that resulted from movement of blood in the body.

Second, from this heartbeat data, a heart rate can be calculated. A single point sensor can also be used (zero-D), or a linear array can be used (1-D), instead of or in addition to the image sensor (2-D). An image sensor would allow this measurement to be seen at many pixels, allowing a heart rate to be determined across an image.

Next, it is not required that the sensor have contact with the subject. The heart rate sensor could be a white LED mounted in an exercise machine, with an image sensor in the display panel of the exercise machine measuring the exercising subject without contact.

Next, the sensor is not limited to measuring the heart rate of a wearer or user. The image could use the same algorithms to extract heart rate from a room full of observers, such as during a poker game or a business meeting, or at an airport checkpoint.

Also, as cardio-workout is defined in terms of minutes of elevate heart rate (either above baseline, or as a percentage of maximum ideal heart rate), one could auto-calculate the minutes of cardio workout in any day, automatically, so that the user does not have to see heart rate graphs or tables, merely seeing just the minutes of ideal cardio-workout per day for example.

Also, from the above example, it is clear that multiple analyses can be performed on different regions of the sensor, allowing multiple people to have measurements such as heart rate measured for each person either simultaneously, or by selection. The approach is not limited to one target subject, nor to the wearer of the device. The determination could be from a glasses-mounted device that displays the heart rate of those around the wearer, and displays these results for the wearer to view.

Next, multiple image sensors could allow such data to be collected from groups of subjects in more than one location, such as from different rooms or different checkout aisles.

Next, note that there is some baseline variation. The size of the pulse signals varies with respiration. Because of this, a respiratory rate signal can be derived, and this can be used to estimate respiratory rate from optical data from wrist, ankle, or face, using measurements obtained even at a distance.

Next, such measurements are not limited just to heart rate. Screening for medical diseases (such as anemia, tachycardia, heart rhythm irregularities, jaundice, malaria, heart failure, diabetes, jaundice), chemical levels (alcohol, high cholesterol), or even fitness can be screened.

Next, because the measures can be broadband, the background light, which varies according to optical contact or coupling of the light to the subject, can easily be subtracted. For example, a baseline may vary widely as a subject runs and moves with a loose fitting heart rate sensor. However, once the baseline movement is corrected (all wavelengths will change, unlike the heart rate measurement which involves only some of the wavelength spectral channels), the background corrected values will more clearly show the hemoglobin variation that represent the changes with heart beats (e.g., heart rate). This allows a non-contact measurement that is resistant to movement, motion, changes in position, changes in background light (such as running in and out of the shadows of trees), all because the broadband values are oversampled, with excess data that allow for background light correction.

Last, because this approach involves broadband light, even background lighting can be used to extract the measures, such as room light in a meeting, or sunlight on athletes working outdoors. This can allow elimination of the white LED.

### Example 2

### Content Aware Detection

As an example of content awareness, one use of the detection of these features is the ability to detect tissue.

Conventional proximity detection involves either an intensity measure that changes as tissue moves closer or farther away, or uses a distance monitoring method to detect the distance from the sensor to the nearest object. Both of these approaches have problems. Both of these methods would view a piece of paper moving closer as the same as a face moving closer. That is, they are neither content-aware nor bio-aware.

In a study performed with human volunteers, a hand was moved over a sensor constructed in accordance with the present invention. The presence of hemoglobin at a tissue saturation level expected in human subjects was used as a measure of the presence of living tissue, and the observed intensity of the signal was plotted as a proximity signal. Also calculated was a pure intensity only signal, which is the standard proximity signal.

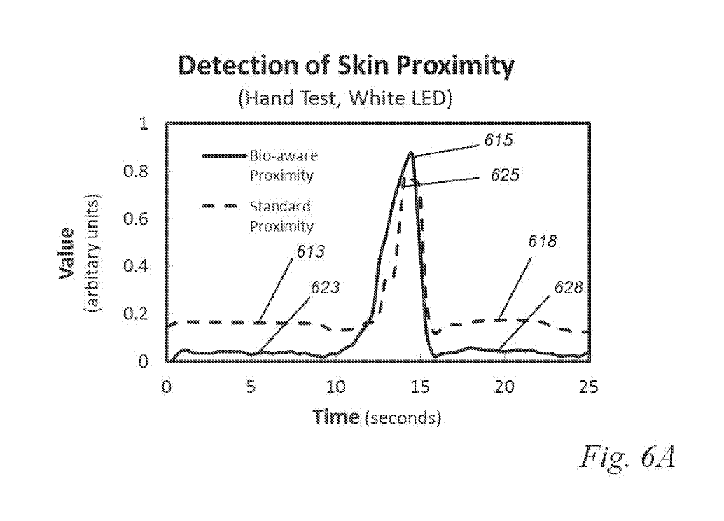

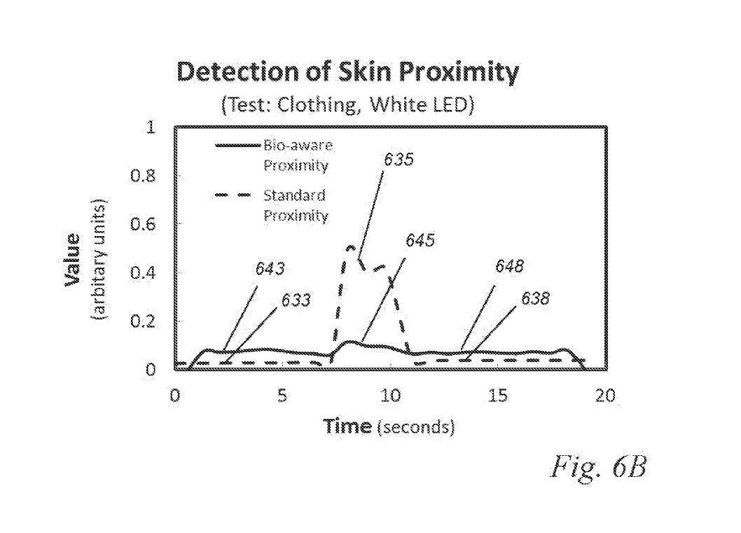

Data are plotted in FIG. 6A-B.

In a first study, data are shown from a hand passing over the sensor, as shown in FIG. 6A. Here, standard proximity signal is shown as a dashed line, starting at a low value before viewing the skin is seen at point 613, then rising to a maximum when the hand is seen at time point 615, then falling again at time point 618 as the hand moves past the sensor. This rise and fall would be consistent with a detection of the hand by a standard proximity sensor. A similar pattern is seen by the bio-aware proximity sensor, starting at point 623, rising to a maximum at 625, and falling again at point 628. In this case, both the standard proximity sensor and the bio-aware proximity sensor return the same result.

Next, the study is repeated, only this time with a piece of inanimate cloth over the wrist passing over the sensor, as shown in FIG. 6B. Here, standard proximity signal is again shown as a dashed line, starting at a low value before viewing the sleeve is seen at point 633, then rising to a maximum when the sleeve is seen at time point 635, then falling again at time point 638 as the sleeve moves past the sensor. This rise and fall would be consistent with a detection of the sleeve by a standard proximity sensor. In this case, with the skin covered, a different pattern is seen by the bio-aware proximity sensor, starting at point 643, failing to rise to a maximum at 645, and remaining low at point 648. In this case, the standard proximity sensor and the bio-aware proximity sensor return different results, because the bio-aware sensor does not detect any living tissue within the field of view of the sensor.

This bio-aware sensing can have many purposes.

For example, a security device could trigger an alarm not just when motion is detected, but when human hemoglobin or a human pulse is detected. This security device could be made to distinguish human hemoglobin from other animal hemoglobin, such that a dog in the security camera view would not trigger an alarm, even if moving. Because the determination can be performed in a non-contact mode at a distance, the technique could be integrated into video cameras, ceiling sensors, lampposts, and the like.

Similarly, the bio-aware sensor could be used to control illumination. In this case, it is not security that is the issue, but energy efficiency. The lights in a room controlled by a motion sensor will turn on when a subject enters, but turn off when the same subject sits still at a computer monitor. A bio-aware device would turn off the lights only when the living human leaves a room, and there is no remaining human hemoglobin or human pulse in the room. Similarly, the lights would not turn on when the family dog enters the room, as the detection would be keyed to human physiological features, while non-human hemoglobin is often spectrally quite distinct from human hemoglobin.

Next, the device could distinguish between real and sham tissue, such as for unlocking security sensors that are image based (such as fingerprint sensors that can be fooled by photocopies of fingerprints).

Hemoglobin is one of the most intense and visible pigments in the body, however there are many other pigments that can be measured by this method.

Fats and water are key body constituents, and have spectral features. Fats exhibit a peak at 920 nm (and elsewhere, including near 760 nm), while water has a peak at 960 nm (and elsewhere, include second differential peaks about 820 nm, large absorbance peaks between 1 and 2 microns, and a broad absorbance peak more or less between 2 and 10 microns).

We constructed a device that measures in the infrared by modifying a commercial spectral monitor (T-Stat 303, described earlier) to measure on the body. This device has a broadband infrared LED instead of a broadband white LED. The broadband infrared LED was designed and constructed for the purpose of having wavelengths above the typical white LED visible range, as shown in FIGS. 3A and B. In this case, light was produced from 650 nm to 980 nm, but any broadband infrared source could be used, including (as discussed under ambient light) sunlight or room light from incandescent bulbs. The spectral peaks were identified using the same fitting methods one would use to fit hemoglobin, such as differential spectroscopy to remove background signal and emphasize the peaks. The concentration of the fat and water was set to 100% by measuring on phantoms containing pure water or fat.

The table below shows determinations from this system, which measuring on a hand, wrist, breast, and head, as shown in Table 1, below:

Next, the device could be used to turn on or off phones when the screen is placed against a face by detection of the human tissue.

Next the sensor could be used to detect where a laptop or tablet is being held, to distinguish human touch from the pressure of a pocket or table.

Last, because different people have differing body composition (fat/water/melanin), different skin thicknesses, different levels of tanning, are of different races, age, gender, and ethnicity, this content awareness could provide some identification features. For example, even without a fingerprint being entered (for instance, if a cell phone is unlocked but is grabbed or picked up by an unauthorized user), then the normal composition of the user in terms of the above characteristics could be used to identify the user, and lock out an unauthorized user who is holding the phone. Similarly, markers (such as dyes, tattoos, unique mixtures of quantum dots) and the like could be used to make very specific optical markers that are nearly impossible to forge, due to the large number of admixtures of different wavelengths of quantum dots (perhaps hundreds could be distinguished) as well as each type having a relative ratiometric concentration, sensitive to one part in 2 raised to the 16^{th} power, or more. As each agent could be in various concentrations, this alone would yield 2 to the 20^{th} mixtures, even without a spatial tattoo patterning. Such implanted dyes could be encapsulated to be stable, providing non-radiowave, optical identification difficult to reproduce or transfer. Combined with a live dead detection, a high level of security could be achieved.

### Example 3

### Heart Performance from a Bracelet Monitoring

In this example, a bracelet was constructed using a white LED light and an optical fiber. The optical fiber allowed for ease of construction, in that a silicon sensor did not need to be incorporated into the small wristband. Rather, the light was transferred from the optical fiber to a commercial spectrally resolved linear sensor and measurement system (T-Stat 303, Spectros Corp, Portola Valley, Calif.) operating in a data-recording mode. This device is a commercial system incorporating a spectrophotometer (Ocean Optics SD-2000+, Dunedin, Fla., USA) to measure light entering the system. Data is recorded on an internal disk, then exported to a USB solid-state drive for storage and analysis, in this case in excel on a laptop computer.

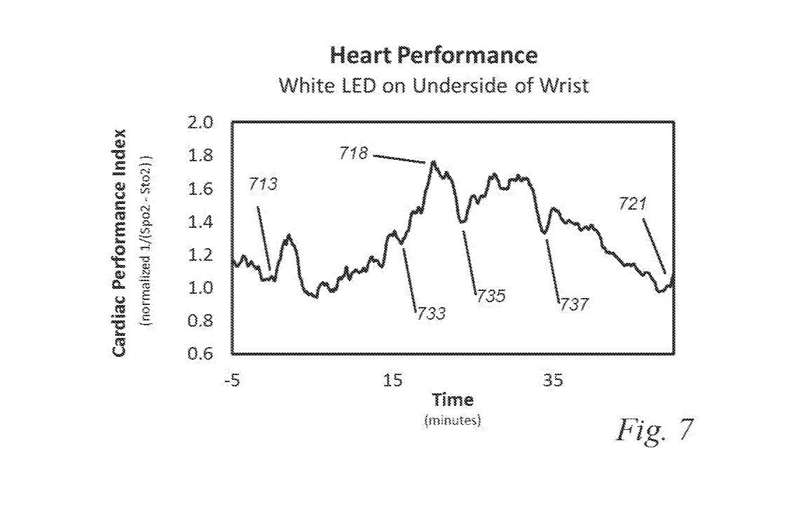

A fit subject was exercised on an elliptical trainer. The power of the workout (joules/hour), the subject's heart rate, respiratory rate, work power, and pulse oximeter reading were recorded using other monitors, including a video recording for synchronization of the various data during analysis. Selected resulting data are plotted in FIG. 7.

In FIG. 7, a measure of cardiac performance is calculated, as the reciprocal of the arterio-venous difference, defined as [1/(SaO2%-SvO2%)]. For this, the SaO2% was estimated using a pulse oximeter, SvO2% was estimated from tissue oximetry of the wrist from data collected from the bracelet using known SvO2% determination algorithms from spectral data, and the data were normalized to 1.00 at the start of the study. The SvO2% measurement was performed using spectral fitting to data from the wristband using tissue oximetry. In practice, a wristband would use the same approach as shown in Example 1, using a white LED, a silicon imager, a spectral filter, and a computational spectral analysis to the spectral data using least squares fit of the spectral data to separate data into component compounds or compound types, such as various forms of hemoglobin, using oxygenated and deoxygenated hemoglobin standards.

Data are shown in FIG. 7. Here, cardiac performance is 1.00 at the start of the study, at point 713. As the subject begins to exercise, performance rises to peak at point 718, then returns to near baseline after recovery to point 721. Also seen are 60-second rest periods at time points 733, 735, and 737. Even during the short rest period, the recovery of heart function is seen. Note also that during this exercise recording, a pulse oximeter readout (medically called SpO2%) remained at 96-98%, and also that the heart rate measured did not recover substantially at all during the rest periods (not shown). There were large dropouts in which the pulse oximeter further was unable to read at all due to motion artifact.

Last, taking the power of the exercise in joules/hour (as measured from the elliptical trainer, which is an estimated workload in this case as this trainer was a commercial exercise device not a physiology lab device, though we expect the power estimates to roughly track a physiology device) and correlating with the cardiac performance on a scatter plot shows that among heart rate, pulse oximeter, and cardiac performance measures, only cardiac performance correlates well with workload (r²>0.82).

There are several points to note here

First, this data was collected with a fiber-based system for ease of laboratory analysis. Use of a mobile system with an LED and a sensor would be one approach to measure these values on mobile athletes. The use of a tethered fiber-optic wrist probe was for proof of feasibility.

Next, cardiac performance could be one of the first performance based devices available to athletes that measures cardiac performance using a simple, optical, non-contact, wrist-based monitor.

The form of a monitoring device includes non-contract pendants, cameras, phones, wristbands, and other wearables. The sensors could be incorporated into clothing such as gloves, spandex suits, caps, bracelets, pendants, and the like.

### Example 4

### Body Composition on a Dieter

Hemoglobin is one of the most intense and visible pigments in the body, however there are many other pigments that can be measured by this method.

Fats and water are key body constituents, and have spectral features. Fats exhibit a peak at 920 nm (and elsewhere, including near 760 nm), while water has a peak at 960 nm (and elsewhere, include second differential peaks about 820 nm, large absorbance peaks between 1 and 2 microns, and a broad absorbance peak more or less between 2 and 10 microns).

We constructed a device that measures in the infrared by modifying a commercial spectral monitor (T-Stat 303) to measure on the body. This device has a broadband infrared LED instead of a broadband white LED to supplement the ambient light present. As noted above, ambient light alone can be used to collect the same raw data, which would be processed in the same manner as shown in this example. The spectral peaks in the detected light were identified using the same fitting methods one would use to fit hemoglobin, such as differential spectroscopy to remove background signal and emphasize the peaks. The concentration of the fat and water was set to 100% by measuring on phantoms containing pure water or fat.

The table below shows determinations from this system, which measuring on a hand, wrist, breast, and head, as shown in Table 1, below:

TABLE 1
Components of living tissue include fat and water. Other substances,
such as volume of bone, collagen, and pigments such as melanin and
heme, therefore the values do not sum to 100%. Multiple measures
around the body could allow for body composition analysis.

| Tissue/Material | Fat | Water |
|---|---|---|
| Finger | 12% | 65% |
| Breast | 45% | 22% |
| Bicep | 15% | 61% |
| Abdomen | 33% | 42% |
| Ankle | 18% | 55% |

This detection of composition is also important, as fats, water, and even proteins in the bloodstream can be measured optically, allowing an estimate of calories taken in by ingestion. Together with calorie expenditure monitoring, taught below in Example 12 and Example 13, this can be used to estimate calorie balance, such as when sufficient or insufficient calories have been ingested in a day (calorie balance), or using the water signal, whether sufficient or insufficient water has been ingested in a day (such as hydration status and water balance).

### Example 5

### Discrimination of an Organic Finger Vs. a Non-Living or Inorganic Sham Finger

Security systems require an identifier in order to detect the presence or identity of a person. Sometimes this identifier is a password or ID chip, while at other times it is a biometric measure (fingerprint, retinal blood vessel pattern). However, some fingerprint detectors can be fooled by something as simple as a cyanoacrylate copy of a fingerprint on cellophane tape.

By performing the analyses of the above examples (detection of heart rate, cardiac performance, fat/water composition), one can easily distinguish real from sham tissue.

In this example, we perform the measures listed in the above example. Tissue is measured for hemoglobin (heme) content. Normal tissue is 20-120 uM heme, with a saturation between 30%-80% for SvO2%. Further, living tissue is mostly water and fat, with water and fat comprising 50-90% of the volume in sum total. Further, there should be a low fitting error (for this algorithm, the error from unrecognized components should be below 200 though this number will vary by system and algorithm). Once these features are taken into account, the real, live tissue (as opposed to dead meat, colored paper, or inanimate objects) can easily be recognized, as shown in Table 2, below:

TABLE 2
Once the components and features of living tissue are taken into
account, the real, live tissue (as opposed to dead meat, colored
paper, or inanimate objects) can easily be recognized.

| Tissue/ | | | | | | | | |
|---|---|---|---|---|---|---|---|---|
| Mate- | | | | Has | | Wa- | Fit | Live |
| rial | Heme | | Svo2% | Pulse? | Fat | ter | Error | Tissue? |
| Finger | 51 | uM | 55% | Yes | 12% | 65% | 68 | Yes |
| Breast | 20 | uM | 71% | Yes | 34% | 22% | 91 | Yes |
| Meat | 450 | uM | 0% | No | 15% | 61% | 122 | No |
| Table | 2 | uM | n/a | No | 0% | 0% | 45341 | No |
| top | | | | | | | | |
| Red | 1 | uM | n/a | No | 0% | 0% | 3911 | No |
| Paper | | | | | | | | |

The “n/a” value indicates no value is determined when the material is not human tissue with blood.

Different subsets of this approach can be taken into account, depending on application. For example, a pulse (heart rate) takes a few seconds to detect, while fat and water can be measured in a microseconds. Therefore, a fingerprint sensor that seeks to verify what is alive and not alive, or real and not real, may wish to use the spectrally determined composition in this analysis.

A few comments on water detection.

Water has a spectrum with peaks that allow detection of concentration. While many combinations of wavelengths can be used, combinations that detect differentiating features of the water spectrum are possible. For example, water has a broad peak at or near 960 nm (peak 1825 of FIG. 18) that differentiates water from the absorbance of fat, hemoglobin with or without oxygen, bilirubin (the pigment of jaundice), and other substances.

One method of detecting water is to look at the difference between the local baseline from 900 to 1000 nm versus the absorbance at the 960 nm peak of water. Analyzing this peak allows determination in Table 2 of the water content. This is translated to a percentage by accounting for the heme and fat components, and normalizing to standards with 100% of each substance in a light scattering medium such as tissue.

Similarly, fat content can be determined using the 920 nm fat peak (peak 1833 of FIG. 18). This peak is often accompanied by a peak near the 760 nm peak of deoxyhemoglobin. A similar peak analysis to that used for water allowed detection of the fat content as shown in Table 2, with normalization as described above.

Hemoglobin can similarly be solved for one or more of its multiple forms. Hemoglobin can similarly be solved for one or more of its multiple forms. There is a double peak for oxyhemoglobin at or near 542 and 577 nm (peak 1842 and 1844 of FIG. 18) and a broader single peak for deoxyhemoglobin at 560 nm (peak 1852 of FIG. 18).

More detailed extractions, such as matrix solutions to multiple simultaneous linear equations can be used as well, though these require more processing by the processor executing instructions stored in memory. Such approaches work for bilirubin (with a peak near 460 nm), alcohol (with peaks above 1 micron), cholesterol with peaks around 1.7 microns), and other pigmented components in the bloodstream.

### Example 6

### Incorporation into Systems and Devices

The sensor as described can be incorporated into a small sensor or device.

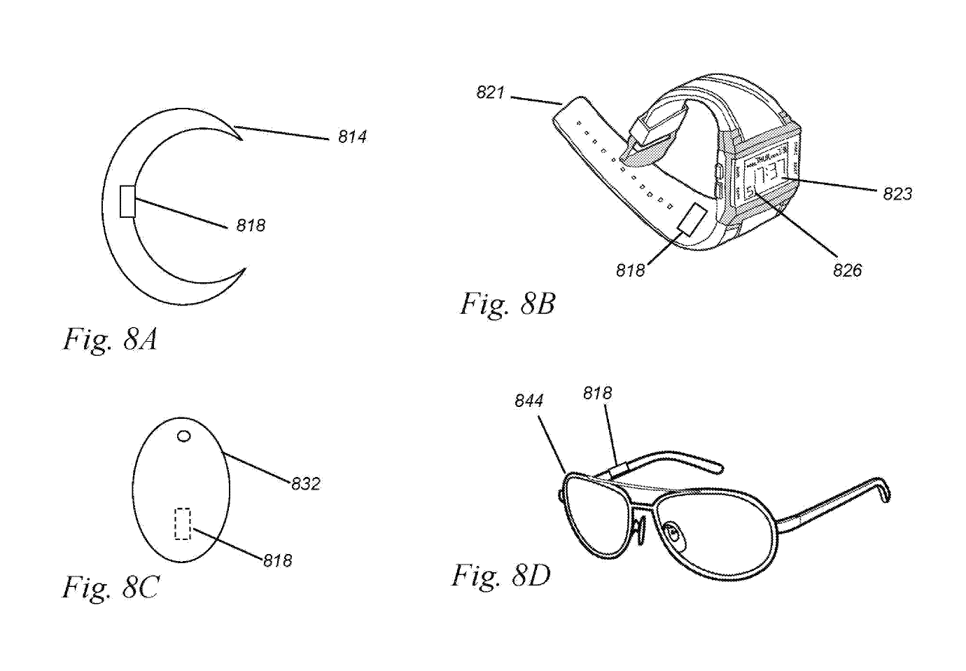

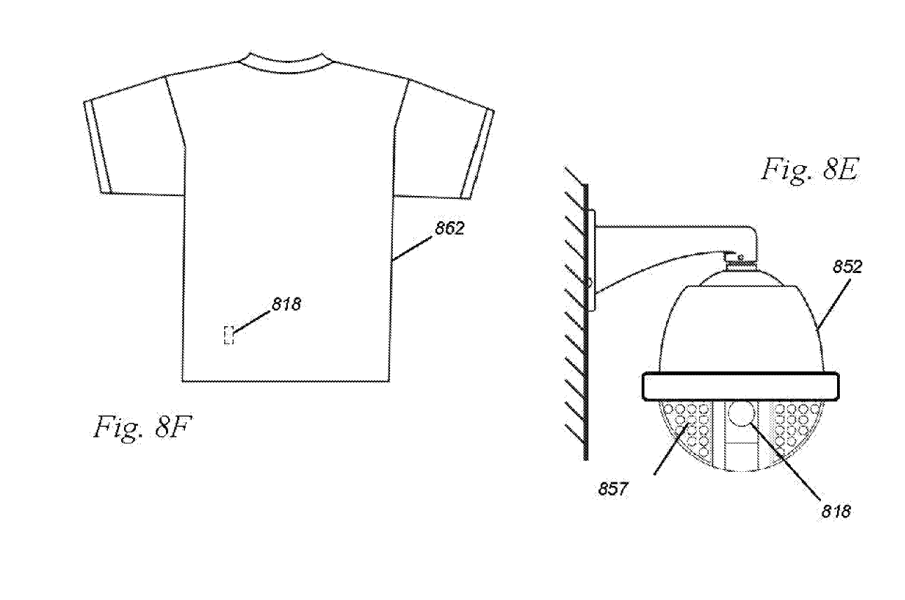

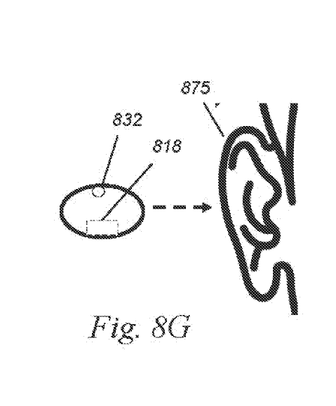

Several devices incorporated into systems are shown in FIG. 8A through FIG. 8G.

A loose fit wristband is shown in FIG. 8A. Here, loose-fit wristband 814 has sensor 818 integrated into its body. This would allow a fitness band, as well as a monitor for persons with chronic medical disease.

A medical or fitness wristwatch is shown in FIG. 8B. Here, wearable watch 821 has sensor 818 integrated into its body or strap. Display 823 shows a user certain useful information, including heart rate 826. This would also allow for a fitness band, as well as a monitor for persons with chronic medical disease.

A heart-rate sensing pendant is shown in FIG. 8C. Here, pendant 832 could hang near the users' body, but not in fixed or permanent contact with the skin, and has sensor 818 integrated into its body. Such sensors could be on two sides, such that one side always senses skin. The proximity sensing and tissue sensing disclosed within could turn on only the side against tissue.

Wearable glasses with sensor are shown in FIG. 8D. Here, wearable glasses 844 have sensor 818 integrated into frame or lenses. A display could be added, much as in heads-up displays to show a user useful information, including heart rate, or into a device such as Glass (Google, Mountain View, Calif.). The sensor can look outward as well, and record heart rates in business meetings, road races, and the like. As noted earlier, the face is a strong source of heartbeat pulses, and the decreased motion compared to the legs and arms makes this an excellent source of measurement.

A remote sensor for ceiling or rooftop mounting is shown in FIG. 8E. Here, remote sensor 852 has sensor 818 integrated into its body or strap. Additional white LED or infrared illumination is provided by LED array 857.

A wearable clothing sensor is shown in FIG. 8F. Here, shirt or textile 862 has sensor 818 integrated into the textile. Wireless communications could be added to communicate with other devices, such as watch 821 or glasses 844 of FIG. 8G, or cell phone 101 of FIG. 1.

An insertable ear probe, into which a heart rate sensor could be placed, is shown in FIG. 8G. Here, earbud 875 has sensor 818 integrated into its body or strap. As noted earlier, the ear is a strong measurement source, though this varies from the pinnae to the auricles to the external canal.

One point of note, different parts of the body have stronger or weaker signals, depending upon what is being sought. For example, the pulsatility at the wrist is often lower than at the fingertips, nail beds, ear lobes, lips, cheek, or forehead, while the ability to measure subcutaneous fat is better over the wrist than in the lips. In contrast, the face has a different venous pulsation with movement than does the wrist. In part, this has to do with the blood flow of the tissue, and the thickness of the skin, but it also is affected by the venous valves present in the arms, but not in the face. Because of this, different sensor configurations, and different algorithms, may be required at different places.

### Example 7

### Non-Contact Sensor Design

In this description the terms loose-fit and non-contact are used. Light forced into tissue (such as from an emitter in physical contact with optical elements of the emitter directly into tissue) and detected by an emitter also in direct physical contact with tissue (such as a CCD pressed directly against skin) travels a different average path than light coming from an emitter source, travelling through the air to skin or tissue, and then scattering and reflecting back to an emitter, also at a distance from the tissue. Further, direct pressure to the measured tissue can suppress pulsatility (though minor pressure may suppress the effects of movement more than the pulsatility).

One way to encourage or ensure the system is non-contact is to place the sensor into a device intended to be kept at a distance, such as cell phone 101 of FIG. 1, or ceiling security sensor 852 of FIG. 8E.

However, such distance is not always possible, especially with wearable devices. In such cases, it may be important at times to force the sensor to remain out of physical contact with the subject, tissue, or object to be examined. In such cases, a design as shown in FIG. 8A through FIG. 8C, or the ear buds of FIG. 8G, may be advantageous.

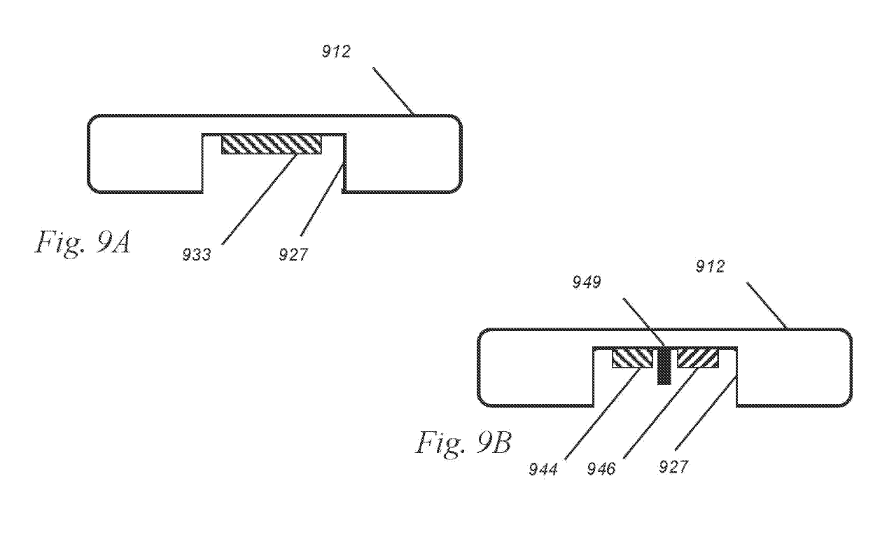

Such a hardware method to ensure the sensor is non-contact is shown in FIG. 9A and FIG. 9B.

First, a recessed non-contract sensor with the illumination and detection on the same chip are shown in FIG. 9A. Here, device 912 has well 927 which holds sensor 933. Well 927 holds sensor 933 away from the skin, by millimeters to centimeters, making light reflect off of the tissue or objects surface when device 912 is held against the tissue or object.

Alternatively, sensor 933 can be separated into separate components, such as emitter 944 and detector 946, with light shield 949 between the two, as shown in FIG. 9B.

Note that in these designs, emitter 944 and/or detector 946 may also each be composed of multiple components that are also similarly separated.

### Example 8

### Measurement of Respiratory Rate

Breathing leads to increases in pulse size at a time constant determined by the breathing rate, as well as shifts in venous blood proportionate to the depth and effort of respiration.

During inspiration (breathing in), the pressure in the chest cavity drops, increasing the rate of return of venous blood to the heart. This in turn makes the pulse volume larger, as cardiac output volume for each beat is driven in part by how much blood returns to the heart during filling during the rest cycle. As a result, the pulse size rises and falls with respiration. This produces a volume change in the total arterial blood signal that has frequency of 8 to 30 times a minute (even faster in infants). By analyzing the average beat-to-beat volume changes in the arterial compartment at longer frequencies than typically seen for heartbeats, a respiration measure can be seen and counted. Averaging for 0.5 to 2 seconds (or frequency filtering) smooths out the pulse, and allows changes in the arterial pulse size to be determined.

Arterial compartment data from exercising human subjects as determined in the previous examples were analyzed using increasing smoothing on the arterial signal, which focuses on the respiratory changes. The respiratory changes can be considered another physiological compartmental contribution (that is, a first compartment with the heartbeat, having a fundamental rate of the heart rate, and a second compartment with the respiratory effect, having with a fundamental rate of the breathing cycle).

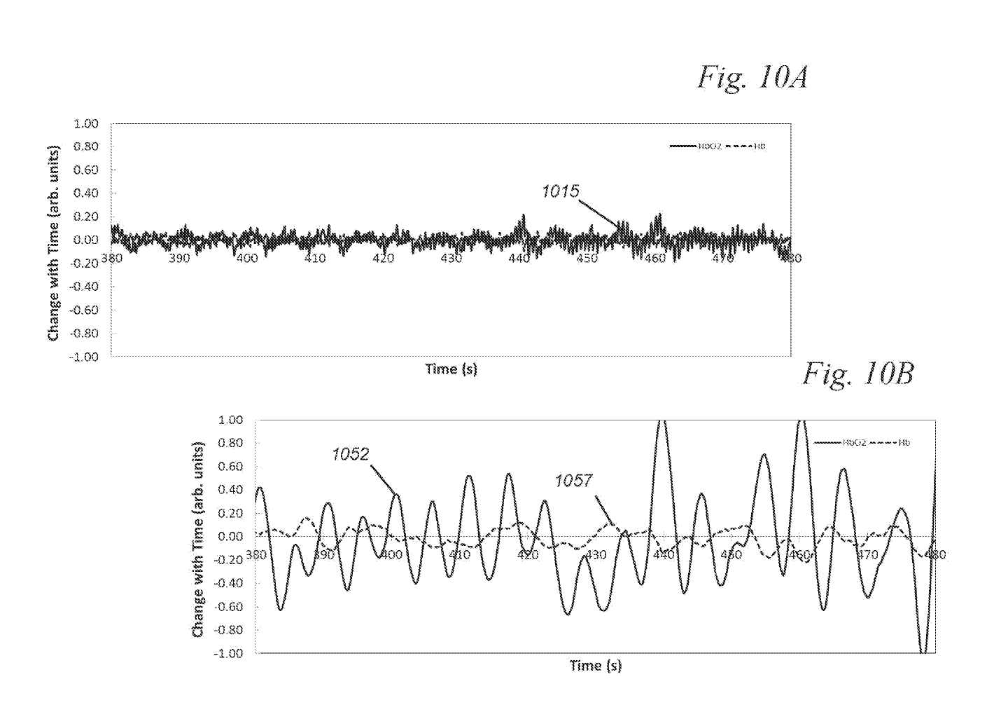

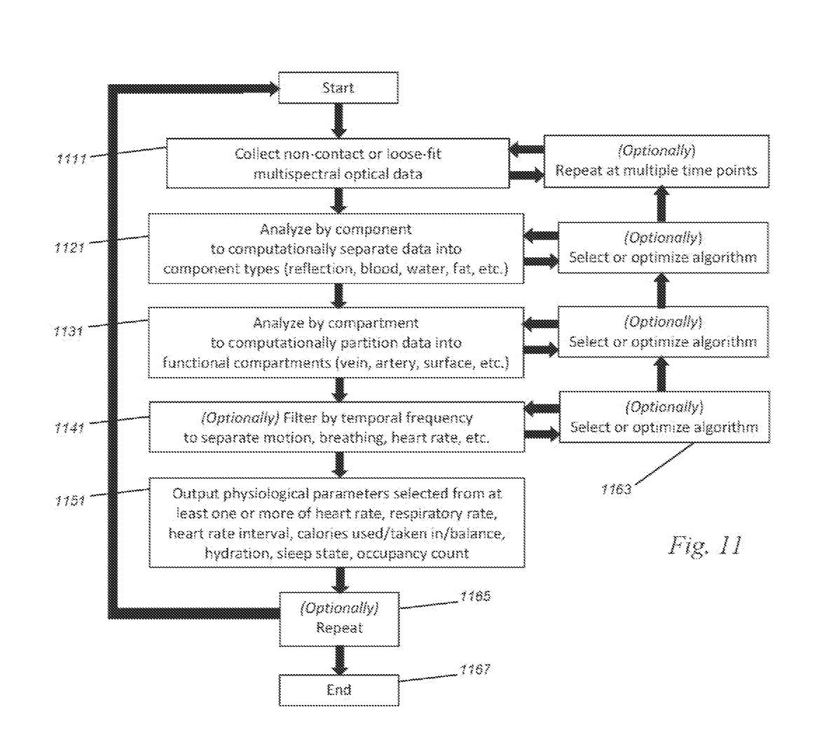

Data are shown in FIG. 10A-B. Here, oxyhemoglobin and deoxyhemoglobin changes over time were initially calculated as in the previous examples. However, different time constants are applied in FIG. 10A and FIG. 10B.

In FIG. 10A, arterial pulse data are shown during exercise (jogging from 380 to 420 seconds into the study) and through the transition to standing still (still from 420 to 480 seconds) in graph 1010. There is little baseline change in the blood because of the previous multi-spectral processing. There are many fine spikes, such as the spikes seen at time point 1015, which represent the heart rate in the arterial signal. These heart rate effects are difficult to see due to the scale, but note that the oxygenated and deoxygenated heme signals are both shown. The time constant for this data is change over a 150 milliseconds with 30 millisecond data sampling.

In FIG. 10B, the same data from FIG. 10A are shown, but subjected to a different time filtering. Here, the data are high-pass filtered with a time constant of 2 seconds, shown in FIG. 10B as graph 1050. Now, the respiratory effect dominates the oxyhemoglobin curve 1052 (solid-line), but is minimally present in the deoxygenated hemoglobin curve 1057 (dashed-line). Counting these cycles shows a respiratory rate of 18 breaths in 100 seconds, or about 14/minute. Further analysis (not shown) into compartments shows the respiratory effect is seen to be isolated to the arterial compartment.

Several Points are Worth Noting in Discussion.

First, these signals can be increased when breathing hard, and therefore the size of the signal increases during hard exercise. The signal is also increased during certain respiratory diseases, such as congestive heart failure (due to pulmonary edema), asthma (due to obstructive pulmonary disease), and choking (due to increased respiratory effort and pressure gradients). One should be able to detect and count coughing, sighs, sneezes, hiccups, and other respiratory anomalies.

Second, by adding another time-constant compartment to the data analysis, the typically 8-30 Hz respiratory signal can be isolated. Similarly, this can be done through Fourier Transform time filtering as well, as is known in the art of time-analysis.

Third, intervals can be used to derive rate, as shall be explored in more detail in a later example. For example, an estimated heart rate (in beats per minute) may be determined as 60/interval, where the interval is expressed in seconds.

### Example 9

### Method

The steps of an exemplary method are shown in FIG. 11.

As noted previously, there are many ways of achieving the steps of this method, but provided a multi-spectral and/or multi-compartmental approach is used to separate the signals in order to produce a stable method insensitive to motion and/or changes in body position, whether in contact or in non-contact modes, these fall within the spirit of the present invention.

A first step is collection of the data, shown as method step 1111. In this invention the data is either non-contact optical data or loose-fit data, with a key feature being that multiple wavelengths are used. For complex determinations, this could be 6 or more wavelengths, but for the purposes of this invention 3 or more is more typical.

Next, the data is filtered. One or more filters may be used.

One such filter is to separate multispectral data into types of tissue, shown as method step 1121. This may be performed using a matrix fit to the coefficients for the various components using published spectral weights, as was shown earlier. Alternatively, partial least squares (PLS), principal component analysis (PCA), or iterative methods could be used in such solutions.

Another such filter is to partitioning the concentrations or features found by multispectral fitting into different compartments, such as partitioning oxyhemoglobin, deoxyhemoglobin, water, or other substances into arterial and venous compartments, shown as method step 1131. In one example, shown earlier, using values of 70% saturation of the venous blood, and 98% saturation of the arterial blood, the oxy- and deoxy-hemoglobin changes can be seen to occur in arterial and venous compartments. This step is described more fully in Example 20.

But there are other phases that can be exploited. For example, there are also venous changes that occur during heartbeats and respirations, with slightly different time constants and phase offsets than the arterial pulse. Also, just as breathing in lowers the intra-thoracic chest pressure, which increases the filling of the heart and produces larger arterial pulses, there can be venous changes as a result of the rising and falling back pressure occurring at the frequency of respiration. Next, body motion, such as raising or lowering an arm, changing body position, or jumping, produces a change in venous blood volume in the tissue (and a smaller arterial change, as arterial blood is higher pressure in muscular arteries, while venous blood is low pressure in floppy vessels). You can see this change by eye when you lower your hand, and your veins become fuller in the back of your hand, while when you raise your hand the vessels collapse and such slow changes are also seen in the studies presented earlier. Because these occur over time, and not instantaneously, there are phases and time constants that can allow identification of additional compartments. Similarly, while the changes that occur with changes in position, or with movement, or with jumping, are largely venous changes, there are some lesser arterial changes, and more sophisticated compartment models may identify these, provided sufficient wavelengths are used.

In each of these cases (heartbeat, respiration, body position changes, movement, and impact from exercise), treating the tissue as having one or more arterial changing component and one or more venous changing components allows for a method of extracting and solving for each of these changes. Each of these compartments is another “unknown” to solve for, and solved by adding more wavelengths. Another unknown, baseline reflection signal, can be solved for using more wavelengths.

Another such filter is to filter in frequency space, such as to separate heartbeat from respirations (effectively two compartments), or even to separate motion (such as probe motion) effects based on their own rhythmic frequencies, as shown in method step 1141. This was shown earlier for separation of heartbeat and respirations using different time constants, but there are many methods such as Fourier Transform or its equivalents to produce a frequency-space data set. Suppression or removal of certain frequency ranges, and back conversion to spectral data would effectively separate the heartbeat and respiratory compartments, and may also be used to remove rhythmic exercise effects, such as walking or running induced probe and body motion.

Finally, data is output in method step 1151. Here, parameters are selected from one or more of heart rate, heart rate interval, heart rate variability, respiratory rate, respiratory depth, respiratory effort, calories expended, calories taken in or ingested, calorie balance, hydration status, time since last ingestion of fluid, step rate, sleep stage, exercise cardiovascular zone, number of heartbeats detected, occupancy count, presence of live or dead tissue, and other physiology measures.

Last, the entire process may be repeated, as shown in method step 1165, or one or more of each of the method steps can be repeated or used to feed back into prior analyses in order to iteratively improve the results, as shown in method step 1163. At some point, the method is ended, at method step 1167. The ending could be a firm end to calculation, or it could be restarted as needed.

Some additional comments on the method.

First, other ways of processing can be envisioned, for example an iterative or more sophisticated model will consider the influence of each compartment on the measurement of the other (such as if the arterial component is NOT 100% oxyhemoglobin).

Second, there are other substances that can be measured. Water, for example, can be measured using water peaks (such as at 960 nm or 820 nm) or any other point provided there is measureable contribution in the absorbance signal from water. Similarly, Ethanol, cholesterol, blood lipids, carotene, even medications can be measured in this manner.

Next, heart rate can be collected as an image, allowing the heart rate to be extracted from multiple persons in an image. Thus, a single point sensor can also be used (0-D), or a linear array can be used (1-D), instead of or in addition to the image sensor (2-D).

Next, it is not required that the sensor have contact with the subject. The heart rate sensor could be a white LED mounted in an exercise machine, with an image sensor in the display panel of the exercise machine measuring the exercising subject without contact.

Next, the sensor is not limited to measuring the heart rate of a wearer or user. The image could use the same algorithms to extract heart rate from a room full of observers, such as during a poker game or a business meeting, or at an airport checkpoint.

Also, as cardio-workout is defined in terms of minutes of elevate heart rate (either above baseline, or as a percentage of maximum ideal heart rate), one could auto-calculate the minutes of cardio workout in any day, automatically, so that the user does not have to see heart rate graphs or tables, merely seeing just the minutes of ideal cardio-workout per day for example.

Also, from the above example, it is clear that multiple analyses can be performed on different regions of the sensor, allowing multiple people to have measurements such as heart rate measured for each person either simultaneously, or by selection. The approach is not limited to one target subject, nor just to the wearer of the device. The determination could be from a glasses-mounted device that displays the heart rate of those around the wearer, and displays these results for the wearer to view.

Next, image sensors could allow such data to be collected from groups of subjects in more than one location, using only the pixels for each subject studied to calculate that subjects physiology data, such as from large rooms, street corners, security lines, or checkout aisles in stores.

Next, such measurements are not limited just to heart rate. Screening for medical diseases (such as anemia, tachycardia, heart rhythm irregularities, jaundice, malaria, heart failure, diabetes, jaundice), chemical levels (alcohol, high cholesterol), or even fitness can be screened.

Next, because the measures can be broadband, the background light, which varies according to optical contact or coupling of the light to the subject, can easily be subtracted. For example, a baseline may vary widely as a subject runs and moves with a loose fitting heart rate sensor. However, once the baseline movement is corrected (all wavelengths will change, unlike the heart rate signal which involves only some of the wavelength spectral channels), the background corrected signal will more clearly show the hemoglobin-varying signal of the heart rate. This allows a non-contact measurement that is resistant to movement, motion, changes in position, changes in background light (such as running in and out of the shadows of trees), all because the broadband signal is oversampled, with excess data that allows for background light correction.

Last, because this approach involves broadband light, even background lighting can be used to extract the measures, such as room light in a meeting, or sunlight on athletes working outdoors. This can allow elimination of the white LED.

### Example 10

### Separation into Compartments

Now, data is further analyzed by blood compartment.

A compartment is a location distinguished by temporal or physiological features that differentiate it from other locations. For example, the skin surface (which reflects and scatters light) can be one compartment. Muscle and tissue is another. The arterial bloodstream is a third example, and it differs in many respects (pressure, oxygenation, compliance) from the venous bloodstream, a fourth example of a compartment. Any region that can be differentiated based on such temporal or physiological characteristics can be a compartment for separation, localization, and computational analysis.

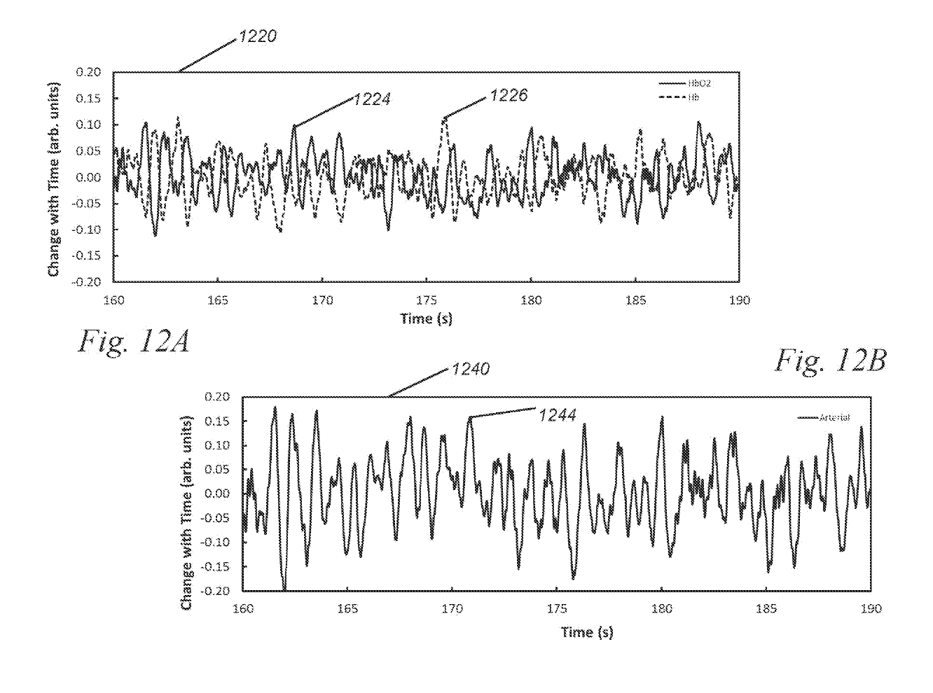

As described earlier, the venous compartment which is affected more by gravity, body position, and impact, while the arterial compartment which is affected more by heart rate and respirations. Separation of these compartments with further analysis is shown as plot 1240 of FIG. 12B. Here, arterial-only plot 1244 is shown.

One key to the compartment separation is that arterial and venous blood have different oxygenation. In this example, we assume that the arterial compartment has a heme saturation of nearly 100%, while the second, venous compartment has an oxygen saturation of 70%. This separation yields an arterial-only volume curve shown as graph 1240 in FIG. 12B. In this graph, the artifacts and noise from body movement and probe movement are nearly gone from the arterial pulse signal. Thus, solving for different compartments therefore allows a pulsatile arterial component, with a heartbeat associated more or less with each of the arterial local maximum values, to be separated from a widely varying venous component. Note that a large change in blood volume and absorbance is only weakly seen visible in FIG. 12A and FIG. 12B, and further that the pulse peaks are clearly seen even at 180 seconds and after, well into movement and/or exercise, in FIG. 12B.

In the calculations of this example, a simplistic but fast way to solve for the compartments was to consider venous blood to be 70% saturated, and for arterial blood to be exactly 100% saturated. Solving only for deoxygenated blood yields changes that must be only venous, as arterial blood has no venous blood in this simplistic analysis. Since venous blood is 30% oxygenated and 70% deoxygenated, the amount of total amount of venous blood changes can be calculated from the deoxyhemoglobin change plus an additional volume change of 30/70th of the deoxyhemoglobin change (that is an additional 30% volume that is oxygenated for every volume of venous blood that is deoxygenated). Removing the oxygenated component of the venous blood leaves a change in this example that must only be the arterial compartment change, which is far more pulse-driven than gravity- and body-position-driven. This allows a pulse to easily be seen, as shown in FIG. 12B.

This approach can be applied to human data collected under study conditions. Multi-spectral analysis of that spectral data, in this case through a matrix solution of simultaneous linear equations, yields the data shown in FIG. 12A-B. Here, plot 1220 of FIG. 12A shows hemoglobin concentration changes over time at the transition from stillness to exercise at 180 seconds, analyzed and re-plotted for 160 to 190 seconds with tissue contact changes and non-heme components minimized by differential analysis, plotted for changes in hemoglobin concentration over time. The oxyhemoglobin concentration (shown as solid line 1224) and the deoxyhemoglobin concentration (shown as dashed line 1226) can be seen to vary differently. These two plots differ in degree of change, timing of peak changes, and even frequency, which clearly demonstrates separation of different signals that change at different times.

Several important things are taught by the above example.

First, it is important to note that such a 70%/100% assumption is not required, and even iterative methods can determine the ratios that best fit the data.

Second, mathematical methods of solving such multiple equations are known. For example, one can apply multiple linear equations, where the values in the equation are: (1) an array of measured data within each waveband, (2) the corresponding absorbances, such as blood with and without oxygen, bilirubin, water, or fat, and (3) the result vector, which yields the concentrations (or changes in concentration) over time. In such an example, if the measured data is an N-element 1-D array named B, representing the data measured at N wavebands, and the known coefficients of effective reflection absorbance (absorbance and scattering) of each of M substances at each of the N wavebands are in a M by N 2-D array (a matrix of coefficients) named A, while the concentrations of each substance to be determined are in an M-element 1-D array of unknowns called X, then the values of X can be determined as (after regularization such that the math works, such as making N=M) then X equals the matrix operation: A⁻¹B. The values for the array of coefficients can be found in publications, or may be experimentally estimated. Alternatively, simple algebra can be used to reduce the complexity of the calculations to mere ratios in certain conditions, or weighted nodal partial-least-squares analysis can be used for even a more complex analysis. All of these fall under the present invention if used to correct for distance and motion in a loose-fit or non-contact physiological monitoring.

As another example, the concentration changes over time can be further partitioned into compartments by time (separation based on frequency, which is different for heart and respiratory variations, for example), or by saturation (the total changes in blood volume and saturation can be analyzed as changes in multiple compartments (such as partition into a venous component of 70% saturation versus an arterial compartment of 98% saturation).

Several comments are now included.

First, it should be understood that the compartment analysis (arterial vs. venous, or gravity vs. pulse) and the substance analysis (hemoglobin, fat, water, skin) can be performed simultaneously, and that they are performed sequentially here for the purposes of clarity of illustration. Further, the analysis can be processed in an iterative manner, which optimizes the separation based on different values of arterial and venous saturation, or upon different time constants for respiratory versus cardiac function.

Next, there are other methods that can be applied to this analysis. Time filtering, such as using a Fourier Transform to place the data into frequency-space from time-space, as is known in the art of data analysis, and can separate a regular heart rate from the pulse effects of respiration, as is shown in a later example.

All of these fall within the scope of the present invention if used in a multispectral or compartmental (or both) analysis to extract non-contact or loose-fit physiological parameters such as heart rate, respiratory rate, R-R heart beat interval, pulse oximetry, or tissue oximetry, cardiac function, bilirubin levels, sweat levels, hydration status, fat/water levels or ratios, cholesterol levels, or the like.

### Example 11

### Rapid and Robust Determination of Rate from Intervals

Measurement of intervals, such as the interval time between peak arterial pulse timing, or the interval time between breaths, is an advantageous method to monitor rates in living subjects.

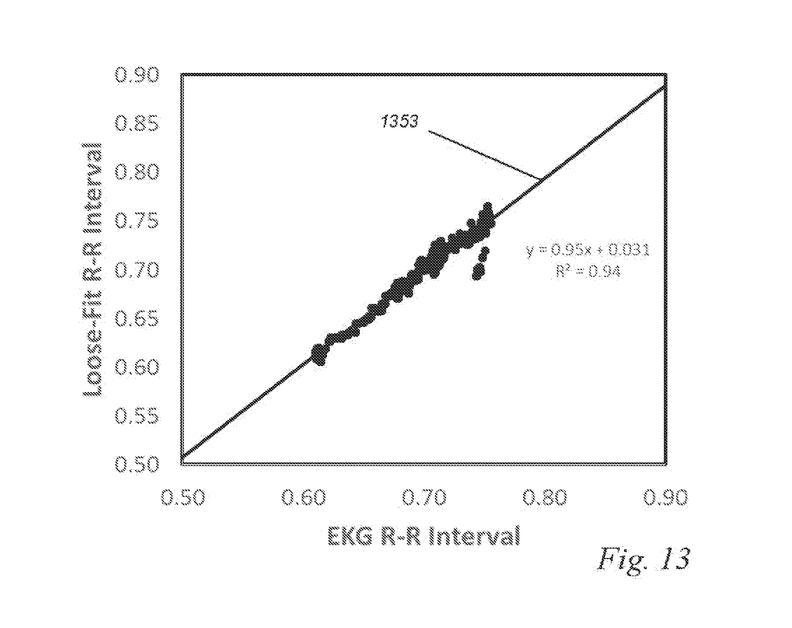

Interval measurement by optical methods correlates well with measurement of intervals via the gold-standard EKG, as shown in FIG. 13. Here, data from another human subject undergoing an exercise protocol and measured by both optical and electrical methods are shown. Plot line 1353 is the best-fit linear plot between the loose-fit arterial compartment beat-to-beat interval, and the electrode-based EKG beat-to-beat heart interval, both plotted in seconds. The plot is very nearly linear, with a correlation (r²) between both measures of 0.94, showing the measure is accurate during exercise. From each of these points, an estimated heart rate (in beats per minute) may be determined as 60/interval, where the interval is expressed in seconds.

Use of intervals in order to determine rate allows for several advantages.

First, consider a heart rate of 115 beats per minute. This would be an interval of 0.52 seconds between each beat, and the heart rate could be estimated by 60/Tᵢₙₜₑᵣᵥₐₗ, where 60 is the number of seconds in a minute, Tᵢₙₜₑᵣᵥₐₗ is the beat-to-beat interval, and the result is in beats per minute.

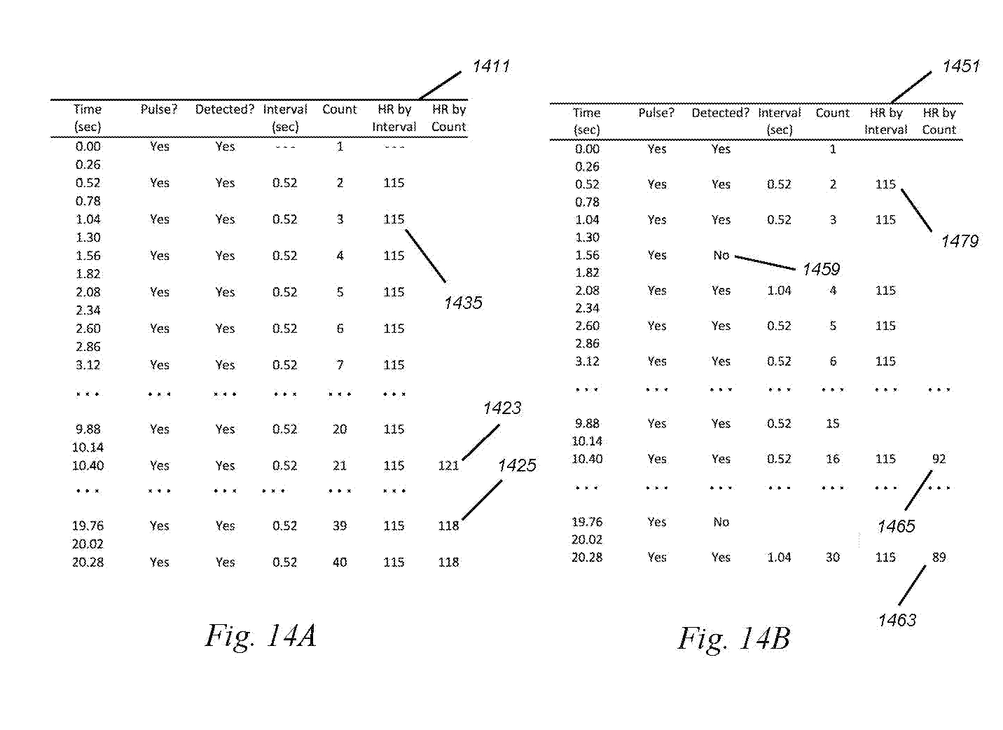

Data accumulates, as shown in FIG. 14A-B. In the case of FIG. 14A, the data are relatively noise free, while in the case of FIG. 14B the data are noisy with data dropouts. Both show model data for a heart rate of 115 beats/min.

In FIG. 14A, data are shown in table 1411. Here, after 1.00 seconds, only 2 heartbeats have been detected; by 10 seconds, 20 beats have been detected. To count to a stable number that estimates heart rate within a few beats per minute, perhaps 20 or 30 seconds would pass, at which time 40 to 60 beats would have been counted since the start. Here, a rate of 123/min is seen in the “HR by Count” column at data point 1423, while a rate of 117/min would be displayed (from multiplying the count of 39 times 60, and dividing by the counting period of 20 seconds) at data point 1425. In contrast, if an interval method is used, a heart rate of 115/min is seen in the “HR by Interval” column after only 1 second has elapsed, at data point 1435, a time when heart rate by counting is blank. The count-based heart rate remains blank as the number of heartbeats (2 beats over 1 second) is insufficient to determine whether the heart rate is 90 (1.5 per second) or 150 (2.5 per second). This difficulty is made even worse if the signal is noisy, as it often is in real world measurements on mobile, active living subjects, as is discussed below.

The ability to determine a rate in 1 second using an interval method represents a significant improvement over counting.

First, the user can receive a heart rate estimate in as little 1-2 seconds or less. In contrast, a runner would have to wait 20 seconds to see the heart rate using a counting system. Anyone who has watched a runner pause for heart rate measurement, and grow impatient standing still, knows that this is significant user experience for athletes and other users.

Second, if the process of measurement requires power, such as driving an amplifier or illuminating an LED, a good heart rate could be determined by interval by having the watch on only a few seconds each minute, as opposed to counting for much longer periods. The impact of this can be estimated. For a wristband with a small watch battery (such as the 25 mAh CR1216-type battery used in the Timex Indiglo, Timex, Conn.), the difference between a 3 mA draw (for a typical LED) occurring only 2 seconds each minute, versus having to stay on nearly constantly for good counting, is the difference between a 250 hour (10½ day) battery life, and an 8 hour battery life.

Third, interval measures are surprisingly robust. Consider a runner with body movement that causes every 4^{th} heartbeat to be missed. This is shown in FIG. 14B. Here, At a rate of 115 beats/minute, the interval measured is first 0.52 sec, then 0.52 sec, but then 1.04 sec including the missed 4^{th} beat in table 1451 at data point 1459, then 0.5 sec again, 0.5 sec, and then 1.0 sec, and repeating this pattern.

By counting, only 3 beats would be seen every 4 seconds, or 90 per minute, as shown by a count of 30 beats in 20 seconds at data point 1463 which is significantly in error, and worse, medically misleading.

In contrast, using the interval method, the modal (most frequent) interval would still be 0.5 sec, for an estimated and still-accurate heart rate estimate of 115 beats per minute at data point 1479. In fact, the 1.0 sec interval could easily be detected as being exactly twice the most frequent rate, and thus clearly determined to be a missed beat double interval. In contrast, the counting method would estimate the heart rate at approximately 90 beats/min regardless of the counting interval. An interval method is thus robust, especially one that uses modal or other filtering.

Of note, there are many ways to estimate intervals. For example, methods to detect cyclic rates such as Fourier transforms, wavelength analysis, and the like are well within the skills on one expert in signal processing.

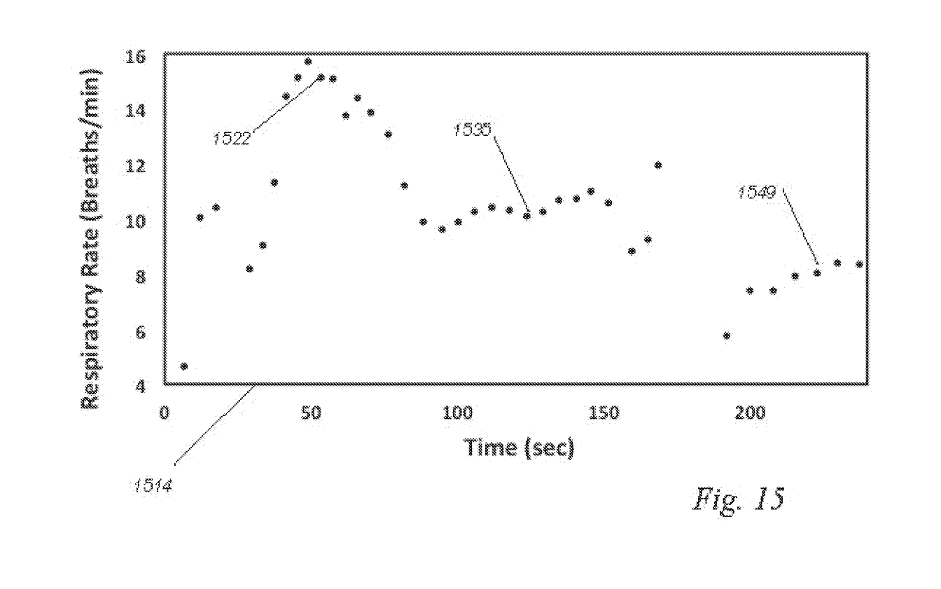

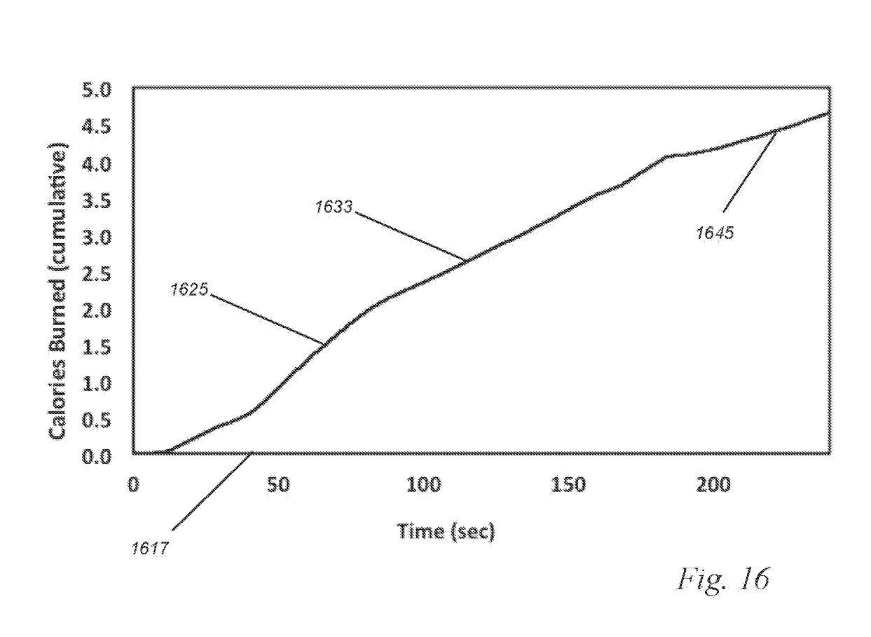

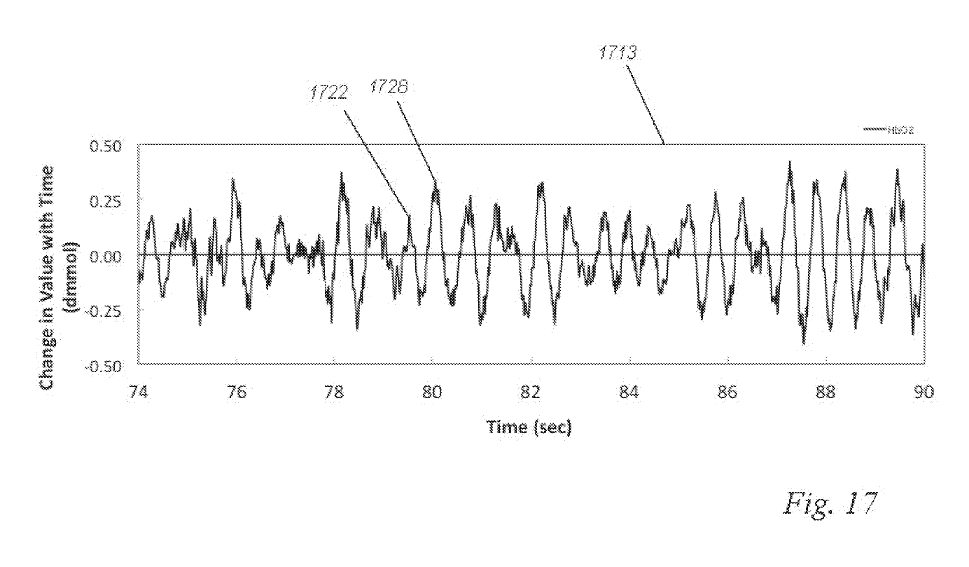

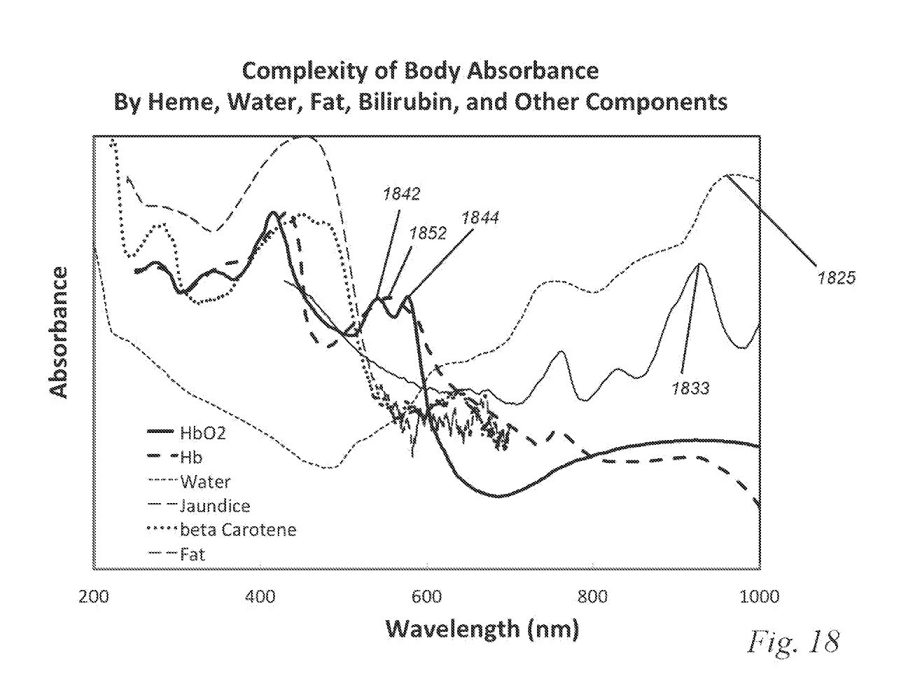

The interval method can be applied to respiratory rates as well. In FIG. 15, respiratory rates determined using an interval method are shown in graph 1514. In human studies, when the respiratory rate was controlled to be 15 breaths per minute, a rate of 15/min was determined by modal interval plotting, shown as time point 1522. When the respiratory rate was controlled to be 10 breaths per minute, a rate of 10/min was determined by modal interval plotting, shown as time point 1535. And last, when the respiratory rate was controlled to be 7.5 breaths per minute, a rate of 7 to 8/min was determined by modal interval plotting, shown as time point 1549.

### Example 12

### Measurement of Calories Used

One of the features that can be measured using this approach is calories, either calories consumed or calories expended. In this example, it is determined in part based on a function of respiratory rate, as derived in the previous example.

Measuring calories consumed is a common laboratory experiment, and is typically performed using the relationship between the calories burned and the oxygen consumed. It is known that in the production of ATP, the energy currency of the eukaryotic cells that occurs in cells, and to a large extend near the mitochondria of the cell, that oxygen is consumed in an electron transfer called the electron transport chain, involving certain enzymes including cytochrome a/a3, cytochrome c, and others. Thus, the basis of calorie measurement in the laboratory is typically a measure of the amount of oxygen consumed, easily measured by flowing a controlled amount of oxygen into an exercise rebreathing setup that uses a closed breathing system.

It is an important realization that in this process, carbon dioxide is also produced. However, in laboratory systems, the carbon dioxide is often scrubbed away, such as by using alkaline agents that react with free carbon dioxide which the carbon dioxide reacts with. While typically ignored this carbon dioxide will become important later.

Another important realization is that the mammalian respiratory rate (at least as well studied in humans) is driven strongly by acidity of the blood and carbon dioxide levels. In contrast, oxygen does not drive respiration, save in certain end-stage lung disease. Humans placed in low oxygen airplanes at altitude will often lose consciousness before responding to their own low oxygen. Our realization includes that because reparatory rate is driven by carbon dioxide more than oxygen and carbon dioxide is produced in proportion to calories consumed, that the respiratory rate is related to calories. The final step is since we have demonstrated how to measure respiratory rate in a noninvasive, noncontact manner, that this measure can be used to estimate calorie consumption in an active, healthy person, such as during exercise using a wearable sensor.

Deriving a relationship between calories used and respiratory rate requires establishing multiple relationships. Some of these relationships have been determined, often for reasons having nothing to do with the real time monitoring of calorie consumption.

Layton (1993) developed new methodology for estimating breathing rates to determine doses resulting from exposure to airborne gases and particles. In this case, calories were not the goal of this research, but rather Layton was looking to develop scales for toxicity. Breathing rates were related to oxygen consumption associated with energy expenditures utilizing a ventilatory relationship that related minute volume to oxygen uptake as given by the equation V=E×H×VQ (where V is ventilation in L/day, E is energy expenditure in kcal/day, H is volume of oxygen consumed in the production of 1 kJ of energy in liters of oxygen/kcal, and VQ is the “ventilatory equivalent”). H is taken to be 0.21 liters of oxygen per kcal based on a 1977-1978 Nationwide Food Consumption Survey (USDA, 1984) and the NHANES II study (US DHHS 1983). VQ is taken to be 27 (unitless) representing the ratio of minute volume to oxygen uptake, a value is derived by Layton from published data of five researchers (Bachofen et al. 1973; Grimby et al. 1966; Lambersten et al. 1959; Saltin and Astrand 1967; Salzano et al. 1984). Layton's equation was later supported by the OEHHA Report (2000).

We want to estimate calories based on respiratory rate. To begin, we modified Layton's equation for our purposes to solve instead for energy expenditure in kcal/min, instead of solving for minute ventilation, as: E=V/(H×VO). By doing this we asking a different question from the investigators interested in calculating respiratory exposure. However, the relationship between minute ventilation and respiratory rate was not clear.

To estimate minute ventilation given a respiratory rate measured by the device, we modified the work of Naranjo et al. (2005) who demonstrated a curvilinear relation between respiratory rate and minute volume expressed by an exponential function. This study recruited trained athletes and tested them on two different treadmill protocols. Expired air was collected and analyzed for carbon dioxide and oxygen, as well as liter flow. From this they determined one relationship between tidal volume, inspiratory and expiratory duration, and respiratory rate. A nomogram was developed for a relation between tidal volume (y) and respiratory rate (x) in this group of trained athletes, with a split by phenotypic gender: y=9.6446 e^{0.9328x} for women, and y=8.3465 e^{0.7458x} for men.

The work of Naranjo addresses only breathing patterns in one group of subjects, but makes no association with calories consumed and the approach fails for subjects breathing at low rates and in non-exercise conditions.

We modified Naranjo's relationships to derive new functions to estimate energy expenditure (in kcal/min) from respiratory rate (in breaths/min) for both men and women. In one example, this relationship was best represented by second-order polynomial equations where the minimum values are the predicted resting metabolic rate, as follows: y=0.0044 x²+0.0798 x−0.2106 for women (r²=0.998) and y=0.0069 x²+0.0463 x−0.0324 for men (r²=0.999). The ability to accurately, non-invasively quantify respiratory rate allows us to combine disparate research to develop a novel solution to measuring metabolism in real-time.

Using these equations, we can now display real-time estimates of calories consumed, using the respiratory rates determined using the method of the previous example, and the calorie conversions as determined in this example.

Results from a human subject are shown in FIG. 16. Here, cumulative calories were calculated, and could be displayed in real time on a wearable watch. A plot of one subject's data is shown as graph 1617. At time point 1623, the subject is breathing more quickly, and this is reflected in a more rapid increase in calories expended, as shown at time point 1625. As the breathing is slowed, there is slower accumulation at time point 1633. Last, at the slowest respiratory rate, the accumulation is slower still at time point 1645.

Several Points of Note.

First, in contrast, some known devices for estimating calories use accelerometers (e.g., Fitbit Flex, Fitbit, San Francisco, Calif.). These devices estimate a calorie consumption using baseline calculations (such as Basal Metabolic Rate, or BMR) from age, weight, height, or other biometrics, and augment those using additional calories based on movement. These devices do not incorporate noninvasive and/or noncontact measures of respiration. And when moving only part of the body, such as when riding a stationary cycle, such devices underestimate calorie use. However, the accelerometers used in such devices could be incorporated into the present device to provide additional, supplemental data to the optical respiration measures within the spirit of the present invention provided that noninvasive and/or noncontact respiratory signals are incorporated into the analysis.

Second, in additional contrast, some other known devices for estimating calories use global positioning (GPS) signals and map data to calculate a distance traveled over time, (e.g., Runtastic, San Francisco, Calif.) and also input such as mode of movement (walking, running, skating, cycling, etc.) in order to estimate calories used. Such GPS and map data could be incorporated into the present device to provide additional data to the optical respiration measures within the spirit of the present invention provided that noninvasive and/or noncontact respiratory signals are incorporated into the analysis.

### Example 13

### Measurement of Calories Consumed and Calorie Balance

In addition to calories used or expended, the number of calories ingested is an important part of the equation. Here, the calculations of Example 4 are relevant. Fat has an absorbance peak at multiple points, including local peaks at 760 nm, 920 nm, and elsewhere. By detecting changes in the peaks of the fat levels, and integrating over time, a measure of the fat calories consumed can be estimated. One exemplary method would be to then assume that fat comprises a fixed amount of dietary calories, and total calories ingested can be estimated as Intake (in kcal or kJ)=Cᵢₙ/F_{fat}, where Cᵢₙ is the estimated total calories ingested, and F_{fat} is the fraction of calories estimated to come from fat.

Once calories used and calories ingested are calculated, a calorie balance over the day can be determined as: C_{bal}=Cᵢₙ−C_{used}, where C_{bal} is the calorie balance over a period of time, Cᵢₙ is the estimated total calories ingested, and C_{used} is the estimated total calories used. In this way, a user could adjust the calories consumed by eating and drinking to balance the calories burned or used during the day.

### Example 14

### Measurement of Hydration

In addition to calorie balance, other balances are important to a user. For example, the water balance could be calculated. Again, using the calculations of Example 4, water concentrations can be calculated. Here, water has absorbance peaks at multiple points, including local peaks near 960 nm and elsewhere (as also shown in the water spectrum of FIG. 18), and second differential peaks near 820 nm. By detecting changes in the peaks of the water levels over time, a measure of the hydration of the subject may be determined.

For example, dehydration will lower the water content at the skin, in the tissues, result in a higher hemoglobin concentration in the blood and capillaries, and reduce the perfusion of the capillaries. In contrast, a drink of water or fluids would, when absorbed, result in the opposite: an increase in the sweat water content at the skin, an increase in the water in the tissues and capillaries, and a drop in hemoglobin concentration in the blood and capillaries, increases in perfusion of the capillaries.

A time since last hydration can be determined, and an automated detection of intake can be determined. One exemplary method would be to then assume that fat comprises a fixed amount of dietary calories, and total calories ingested can be estimated as Intake (in kcal or kJ)=Cᵢₙ/F_{fat}, where Cᵢₙ is the estimated total calories ingested, and F_{fat} is the fraction of calories estimated to come from fat.

### Example 15

### Ambient Light

As an example, the hemoglobin pulse is shown from a signal collected in ambient light in FIGS. 2C and 2D. An embodiment of the present invention was made omitting white LED 103 shown in FIGS. 2C and 2D. In addition, an embodiment was made in which the software was able to shut off white LED. Both of these systems achieved a reduction in power, with current dropping over 2 mA, for a savings of 10 mW at a 5 v LED drive voltage from having the white LED off

Data were collected from the hand of a human subject at a distance of approximately 10 cm, in order to allow the room light to reach the skin and eliminate any shadow from the sensor board over the target sample tissue site.

The signal is clearly visible as peaks (for example, peaks 1722 and 1728) where collected from distance of 10 cm from the subject in ambient light. Such signals can be processed as described in earlier examples to separate signals into various compartments and determine pulse and respiratory rate, such as is illustrated in the flow chart of FIG. 11. Depending on the number of wavebands selected, and their range, such signals can be used to extract heart rate, respiratory rate, heart rate variability, respiratory rate, calories, hydration, sleep state (based on rate and variability), even blood alcohol or blood fat levels.

### Example 16

### Sleep Stage

Many sleep-stage bands collect accelerometer data. Such devices determine sleep stage by motion, which can be very inaccurate. In contrast, heart rate, heart rate variability, and respiratory rate also fit into these equations. Once a good measure of heart rate, heart rate variability, and respiratory rate is obtained using the methods described herein, sleep stage can be extracted using the equations and methods from the published literature. More accurately, a database can be assembled using remote monitoring from the optical devices disclosed herein, and the features extracted can be used to determine sleep stage using any depth of sleep algorithm known in the art.

### Example 17

### Complexity of Body Absorbance

The complexity of light absorbance in the body is not straightforward, which is one reason that use of a limited number of wavelengths will fail to correct for the many substances in the body, particularly if there are rapid changes in absorbance caused by drifting LED lights (less of an issue with filter-coated detectors and broadband light sources).

For example, with regard to FIG. 18, here we show the spectra of just a few substances in the body, including water, bilirubin, hemoglobin proteins with and without oxygen, fat, and water. Use of spectral analysis, such as simple peak size detection to multispectral fitting, can allow these various components to be separated. In general, unless a method can be found to suppress a signal (such as using time-varying pulsatility to focus on certain compartments such as the bloodstream, or saturation-separation to focus on arterial vs. capillary vs. venous compartments, or use of wavelengths where the spectral contribution of the interfering substances can be minimized), the signal remains complex.

Here, the peaks of water, fat, and hemoglobin have been described earlier. For example, water has a broad peak at or near 960 nm (peak 1825) that differentiates water from the absorbance of fat, hemoglobin with or without oxygen, bilirubin (the pigment of jaundice), and other substances. Similarly, fat content can be determined using the 920 nm fat peak (peak 1833). This peak is often accompanied by a peak near the 760 nm peak of deoxyhemoglobin. Hemoglobin can similarly be solved for one or more of its multiple forms. There is a double peak for oxyhemoglobin at or near 542 and 577 nm (peaks 1842 and 1844) and a broader single peak for deoxyhemoglobin at 560 nm (peak 1852). Such approaches work for bilirubin (with a peak near 460 nm), alcohol (with peaks above 1 micron), cholesterol with peaks around 1.7 microns), and other pigmented components in the bloodstream.

The same approaches that allow determination of solutions of equations or functions that produce concentrations for water, fat, and hemoglobin can be used to extract spectral information from other substances at other wavelengths, including proteins, DNA, alcohols, chlorophyll, and other pigmented substances. The wavelengths required for analysis can be in the ultraviolet, visible, or even infrared wavelengths, provided that spectral features exist allowing extraction of concentrations or solutions to equations that are a function of the presence, absence, change, concentration, or variance in those substances over time.

### Example 18

### Skin-to-Sensor Distance Change with Movement

Just as a normal heartbeat leads to a pulsatile, rhythmic increase in the amount of arterial blood in certain tissues (and thus an increase in the absorbance of light, as shown in the prior example), other events can also significantly change the amount of light reflected by a tissue such as skin. For example, merely moving the skin on which a light shown farther away or toward a sensor will change the amount of light returning from the skin tissue.

We constructed a research probe that allowed the sensor shown in FIG. 2D and a light source to be attached to a loose wristband, with data collected at many wavebands, in accordance with the present invention. This research probe allowed measurements to be collected over a wide range of wavelengths. Data were then collected with this system on a human subject with a sensor placed within 1 cm of the skin. Then the sensor was moved away from the skin, then toward the skin again, and this cyclic movement was repeated for a total of 3 cycles.

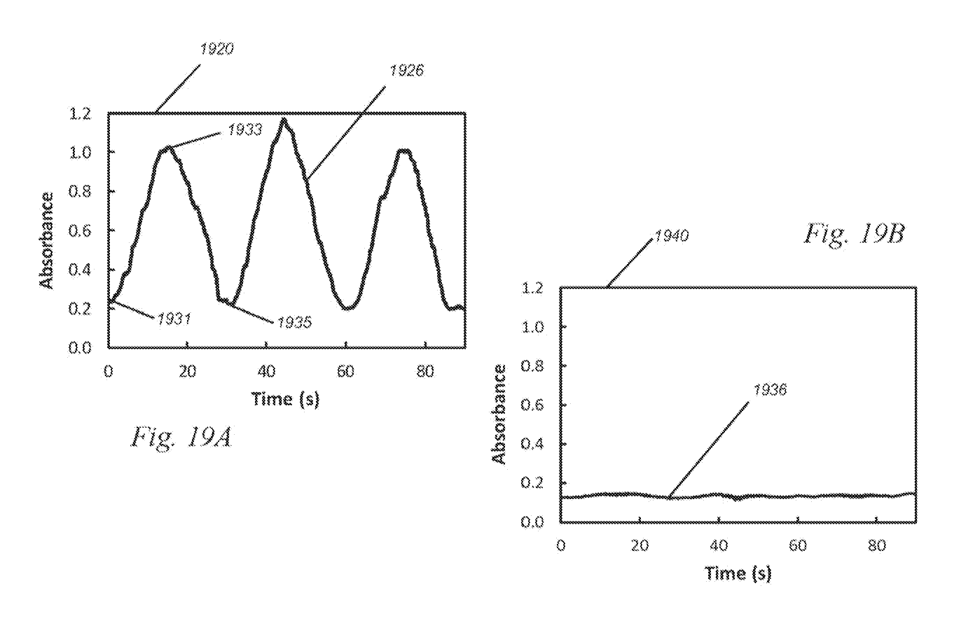

Data from this study are shown in FIG. 19A-B. The 3 movement cycles are visible in graph 1920 of FIG. 19A as plot line 326, where the absorbance of light is plotted relative to a reference standard (in this case, conventional foamed open cell Styrofoam, known to provide similar scattering to tissue with an absence of spectral features). Here, absorbance begins at a low at time point 1931, rises to a local maximum as the sensor is pulled away from the subject's forehead at time point 1933, the falls again as the probe as moved closer again to another local minimum at time point 1935. This pattern in the data is seen to be repeated twice more, for a total of movement through 3 absorbance cycles.

Note that the movement of the probe away from, then back toward, the subject's skin produces an apparent change in total absorbance in this single-waveband plot (e.g., data are plotted using just one color band such as 560 to 570 nm, or after measuring just one intensity across all colors in a camera sensor over time). This matches the number of movement cycles in the study.

Importantly, this cyclic pattern caused by the movement in FIG. 19A is in many ways similar to the cyclic pattern caused by the heartbeat seen previously in FIG. 5A-B. In fact, if the subject's heart rate were about 60 beats per minute, and someone was jogging with a loose-fit sensor such that the cyclic movement of the sensor occurred at a similar frequency, the pulse curves of FIG. 5A-B might be virtually indistinguishable from the body movement intensity curve in FIG. 19A. Worse, if the jogging rate and the heart rate were different, it might be difficult to determine which is the pulse and which one is the distance movement when using just this one single-waveband plot line (this is shown at one wavelength only, but when adding additional wavelengths in accordance with the present invention the problem is solved, as shall be shown).

Because hemoglobin can be determined using spectroscopy at multiple wavelengths, and the spectrum of the skin by itself is different than the spectrum of blood, multiple linear equations can be solved to partition the signal into blood and into skin contributions. In this example, we use the fact that hemoglobin absorbance is 100-fold higher at in the 500-600 nm range than it is in the 650-700 nm range, whereas the scattering of skin is more nearly equal over that range. By relying upon the differing absorbance of each tissue at different wavelengths, a multi-wavelength system allows separation of the signal into blood and skin tissue quantities, or even into oxygenated, deoxygenated, and non-blood tissue quantities.

The result of this multispectral approach is shown in the results shown in graph 1940 of FIG. 19B. When skin correction is performed using additional wavelengths at which hemoglobin is not significantly absorbed, and the effect of skin proximity is calculated and removed from the data using multispectral analysis, the results look very different than those seen in FIG. 19A. Here, plot line 1946 shows that after removing skin and distance effects, the movement artifact is reduced by nearly 100-fold, and only small variations remain (not even large enough to even show well in this plot). The remaining smaller temporal variations can be used to extract heart rate, as will be shown in later examples. Addition of even more wavelengths, selected for their ability to discriminate between blood and skin, improve the separation even more, such as with correction for body position changes, as is discussed next.

In some cases, reduction of the noise by half (an improvement in signal to noise of “one bit”) may be sufficient. In this case, the reduction is by more than 90%, or roughly 7 effective bits of signal to noise improvement.

### Example 19

### Body Position Change

Again, just as both the heart beat pulse and probe movement each lead to a change in the amount of various components of the bloodstream (in these examples, blood and water), and thus leads to changes in the absorbance of light, positional changes of the body are yet another factor that change the amount of light returning from the body.

For example, by merely raising your arm above your head, or by lying down then standing up, one changes where the blood redistributes in the body (this is a big issue in space travel, where the blood that is normally in your legs due to gravity distributes everywhere, making your face puffy and engorged with blood). One can see this effect by dropping one's wrist at one's side, and noting the swelling up of the veins (with no similar effect easily seen on the arteries), and then raising one's hand above one's head, and noting the emptying of the veins. There is a reason for this: arteries are high-pressure, muscular vessels with little change in volume with pressure (in physics terms, arteries have a low compliance, defined as change in volume with pressure), while veins are floppy, baggy, low-pressure tubes with a large change in volume with a very small change in pressure (high-compliance). A shift in the location of various components of the bloodstream between the veins, arteries, and capillaries creates a signal that can mask the more subtle changes introduced by the beating heart and by breathing.

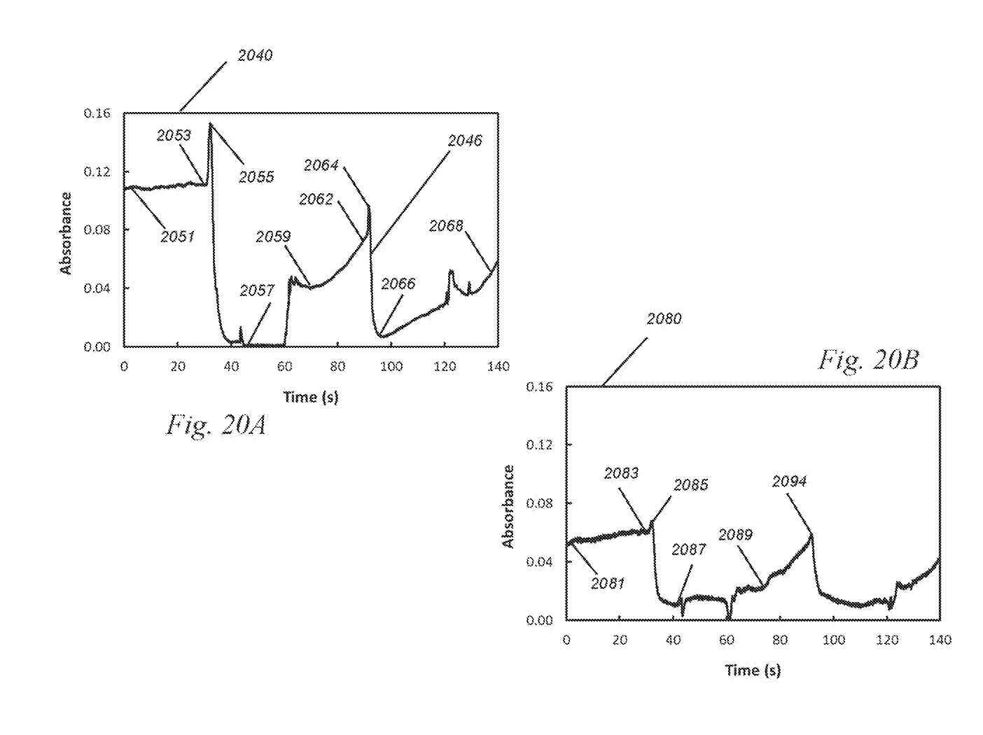

Data collected using the system of the previous example is shown in FIG. 20A-B. In this study performed on a human subject, a sensor was placed within 1 cm of the skin of the wrist, but the light emitter and the light detector do not touch the skin because the light source and detector are recessed in the probe (for example, as is shown in FIG. 9A-B). During the study, the subject is held still and stable for 30 seconds, then the wrist is moved up in the air above the head and held for 30 seconds, then brought back to waist height and held for the seconds, and this movement cycle is repeated for 1 additional cycle.

These 2 movement cycles are visible in graph 2040 of FIG. 20A. Here, the absorbance of light is again plotted relative to a reference standard as plot line 2046. The absorbance begins at a high at time point 2051, representing data when the wrist has not be raised and has been in the same position for several minutes, such that the absorbance remains stable through time point 2053. Next, the data spikes at point 2055 then falls rapidly to a local minimum at time point 2057 as blood drains from the wrist, then rises again as the wrist is once again raised at time point 2059, continuing to rise through point 2062, then spiking again at point 2064 and falling again at time point 2066 as the wrist is again dropped though rising slowing by point 2068.

Several points are important to note.

First, a loss of signal (increased absorbance) with moving away from the skin makes intuitive sense. If bodies remained at rest, then such measurements could be straightforward. But when considering only one wavelength, it is difficult to determine whether a change in intensity is a change in the proximity or contact with skin, or a change in blood volume in the tissue, or a change in the blood content from a heartbeat. More violent movement, such as impacts during running and jumping, product strong changes that make heart rate detection very difficult to perform accurately at one wavelength, except in certain circumstances or with addition of additional monitoring data.

Second, the same pattern (falling with raising of the wrist, rising with lowering of the wrist) repeat each cycle, showing these general changes are a result of body movement. While a moving probe can be corrected with a tight wristband or well stabilized probe, the body will move in position during exercise, making this change difficult to correct for. Many commercial probes correct for this by being not only fixed in place with a strap to prevent probe movement and ambient light seeping under the sensor, but also are sufficiently tight so as to reduce venous blood flow. Such approaches cannot be used in a non-contact loose-fit or remote monitoring device, and they fail under such circumstances with movement.

Third, a rising and falling pattern is the same type of signal produced by the heartbeat, which can make the signals hard to separate if the body motion and movement is rhythmic and occurs at a rate that a heartbeat would be expected to occur (such as a once a second movement from footfalls during running) The size of the absorbance change with movement is on the order of 0.05-0.15 absorbance units. This is 100 fold larger than the changes due to the heartbeat. As changes in body position are common during jogging and other exercise, and if rhythmic can be very similar to the heartbeat curve seen in FIG. 5A-B, the large size presents additional barriers to uncovering the heartbeat.

Using multiple wavelengths, the same correction for changes in distance to the skin shown in Example 18 was performed, and the data as shown in FIG. 20A is re-plotted after correction, as shown in graph 2080 of FIG. 20B. Unlike in Example 18, the skin correction does not eliminate most of the large swings in absorbance. In fact, the absorbance still begins high at time point 2081 (compare time point 2051 in FIG. 20A), still spikes at point 2085, then falls rapidly to a local minimum at time point 2087 (compare time point 2057), rises again at time point 2085 (compare time point 2055), falls again at time point 2087 (compare 2057), and rises at time point 2089 (compare time point 2059) as the wrist is again dropped.

As before, reduction of the noise is by more than half (absorbance changes up to 0.15 in FIG. 20A, but only up to 0.05 in FIG. 20B, an improvement in signal to noise of 1 to 2 bits). Such an improvement may be sufficient for certain applications.

So, it may be asked why didn't this skin correction work in the same way in this example as it did in Example 18. The answer has to do with physiology of compliance. When one puts one's hand down low, the blood distributes by gravity into the arm and the absorbance increases. This represents not just a change in skin contact and distance, but an actual change in the blood content of the measured skin as well.

To solve for blood changes, one needs to solve for the presence of blood (or water), or in more detail solve for the presence of oxy- and deoxy-hemoglobin. When just the skin effect is considered, this totals either 2 or 3 unknowns without separation into compartments.

In general, the number of unknowns to be solved for means that at least the same number of equations are needed to solve it well (in mathematics, it would be said N wavelengths are needed to solve for N unknowns, in order to not be underdetermined). Our biggest unknowns so far are the amount of hemoglobin and skin reflection/scattering, which requires at least 2 wavelengths. In order to determine oxyhemoglobin, deoxyhemoglobin, and skin, at least 3 wavelengths are required to solve this data set well. This is a simplification, as arteries have both oxygenated and deoxygenated blood, and there are other substances that absorb light. But there are also wavelengths were water absorbs well, so a pulse could come from the water signal instead of the amount of hemoglobin. In the next example, it will be shown how blood movement, as opposed to the probe movement, can be more completely corrected.

It is worth noting that while the predominate change in the data in FIG. 20A-B is a blood volume change, there does appear to be certain changes that are due to contact with the skin distance as well. Note the upward spike at point 2055 in FIG. 20A, which occurs when the wrist is thrust high into the air. This change is not only in a different direction than the fall in absorbance that occurs with blood draining from the arm after a raising of the wrist, but it also has a different time component. However, correction of the skin changes in FIG. 20B shows that the spike at the same time point is nearly gone after skin correction at time point 2085. This suggests that the spike at the start is not a blood change, but rather movement of the loose fit bracelet. A similar spike appearing in FIG. 20A at time point 2064 is also nearly gone in FIG. 20B at time point 2094. This suggests that a multi-wavelength correction may be required during physical exercise and movement as both skin and blood distribution changes will occur with motion.

In the next example, it will be shown how movement of blood in the body can be corrected for, and used to enhance the heartbeat signal.

### Example 20

### Rejecting Blood Movement Using a Compartment Model

So how does one solve correct for blood movement, given that water and hemoglobin are present in all the compartments? The answer is to consider physiology.

Movement of blood during body movement tends to occur in the veins. This is because veins tend to be floppy, thin, low-pressure tubes that are partially distended with blood, and therefore swell and empty small changes in pressure, such the column of pressure created by gravity. In contrast, there is a much smaller change in the arteries. Arteries are thick and muscular, and are already under substantial blood pressure. Therefore, when the body moves, gravity does not cause them to empty or fill very much. Because movement under gravity occurs more in the veins than in the arteries, this allows multi-wavelength analysis to include another “compartment” in the analysis: what is that some of the oxyhemoglobin and deoxyhemoglobin is in the veins, and some is in the arteries.

Now, if arterial and venous blood were identical in composition, this floppy versus stiff tube approach would not add much useful information. However, arterial blood and venous blood different in many important ways: pH, oxygen content, dissolved carbon dioxide, and other ways. Venous blood, for example is typically 70% oxygenated in healthy adults at sea level (that is about 70% oxyhemoglobin, 30% deoxyhemoglobin, not including smaller amounts of other heme forms typically totaling under 2% of the hemoglobin). At the same time, arterial blood is typically about 95-99% oxygenated in healthy adults at sea level (that is, about only 1-5% deoxyhemoglobin, and the rest is oxyhemoglobin, again not counting other heme forms present).

These physiological and compartmental differences in oxygenation allow the measured components to be sorted into multiple compartments (e.g., arterial, venous, skin, muscle, gut, and liver). For example, skin is where melanin and other pigments not typically seen in blood are concentrated, while muscle is where myoglobins are typically found. In contrast, hematin, a form of hemoglobin found in malaria victims, is typically found in red blood cells in the bloodstream.

Now, rather than use just a few wavelengths, we can determine a heart rate from data collected 30 to 100 times a second from a spectrally resolved system with 6 to 8 wave bands, to which we will apply a method of multi-compartment multi-spectral analysis.

Data were collected using the research system of the of the previous example on a human volunteer undergoing exercise protocol that consists of a series of actions performed for 1-3 minutes each: sitting, abruptly moving arms while sitting, standing, abruptly moving arms while standing, squats, jogging or jumping in place, standing, then sitting. This subjected the sensor to movement of the probe as well as to changes in body position.

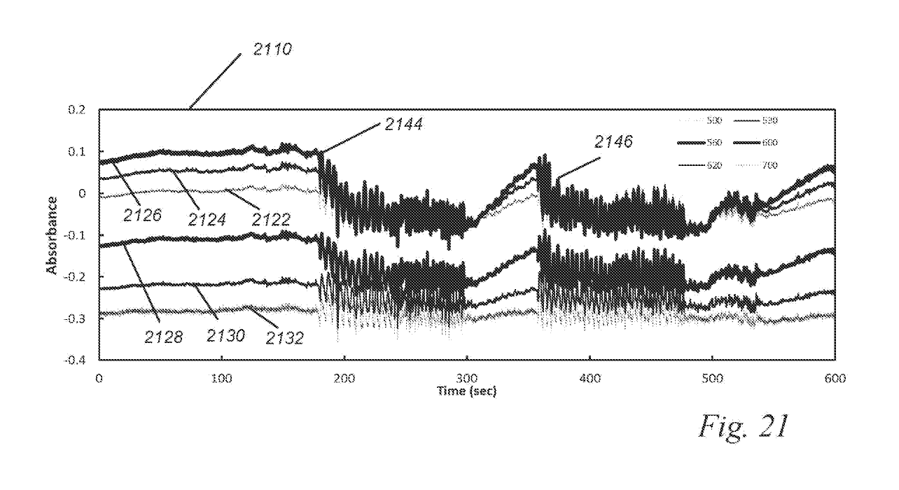

FIG. 21 shows absorbance at 6 wavebands over 600 seconds during the exercise protocol described above, as compared to a reference standard. Plots for wavebands in the region of 500, 530, 560, 600, 620, and 700 nm are shown over time as plot lines 2122, 2124, 2126, 2128, 2130, and 2132, respectively. These wavelengths are shown for reasonable detection of hemoglobin, but also for best separation on a graph for illustration purposes. Those skilled in the art would be aware algorithms can be optimized for reduced noise, such as by selecting combinations of wavelengths that best discriminate between tissue, oxyhemoglobin, and deoxyhemoglobin (or whichever substances are of interest).

Note the wide variation in the signal with movement of the body and probe during exercise in

FIG. 21. For example, a period of relative physical stillness from 0 to 120 seconds shows relatively stable measures. During this period, the thickness of the plot lines 2122, 2124, 2126, 2128, 2130, and 2132 comes from the heartbeat, respirations, normal physiological changes, and some background noise (there are minor differences as well due to the plotting of the lines at different widths as well, in order to allow the plot lines to be distinguished by eye in the figure). In contrast to the early quiet period over the first 120 seconds, the period from 120 to 180 shows additional fluctuation as the arms are moved, and large changes during movement, such as the transition from stillness to exercise and the transition from one body position to another For example the movement at 180 seconds into the study at time point 2144, and at 360 seconds into the study at time point 2146, each produces large changes in the raw signal.

After correcting for the movement of the probe relative to the skin, as shown in previous examples, then multi-spectral linear equation analysis at these 6 wavelengths allows both oxygenated and deoxygenated hemoglobin levels to be determined, in addition to changes in skin distance. For such analysis, 3 or more wavelengths are required to separate the 3 unknowns: tissue, heme with oxygen, and heme without oxygen signals. With multispectral data, one way to process the data is to use multiple equations with multiple unknowns, such as linear matrix fitting, an approach known to those skilled in the art

Multi-spectral analysis, in this case through a matrix solution of simultaneous linear equations, yields the data shown in FIG. 12A-B. Here, plot 620 of FIG. 12A shows hemoglobin concentration changes over time at the transition from stillness to exercise at 180 seconds, analyzed and re-plotted for 160 to 190 seconds using the same data plotted in

FIG. 21, only here with tissue contact changes and non-heme components minimized and plotted for changes in hemoglobin concentration over time. The oxyhemoglobin concentration (shown as solid line 1224) and the deoxyhemoglobin concentration (shown as dashed line 1226) can be seen to vary differently. These two plots differ in degree of change, timing of peak changes, and even frequency, which clearly demonstrates separation of different signals that change at different times.

Now, data is further analyzed by blood compartment. As described earlier, the venous compartment which is affected more by gravity, body position, and impact, while the arterial compartment which is affected more by heart rate and respirations. Separation of these compartments with further analysis is shown as plot 1240 of FIG. 12B.

The key to the compartment separation is that arterial and venous blood have different oxygenation. In this example, we assume that the arterial compartment has a heme saturation of nearly 100%, while the second, venous compartment has an oxygen saturation of 70%. This separation yields an arterial-only volume curve shown as graph 1240 in FIG. 12B. In this graph, the artifacts and noise from body movement and probe movement are nearly gone from the arterial pulse signal. Thus, solving for different compartments therefore allows a pulsatile arterial component, with a heartbeat associated more or less with each of the arterial local maximum values, to be separated from a widely varying venous component. Note that the large change in blood volume and absorbance seen at 180 seconds in

FIG. 21 is now gone, and only weakly seen visible in FIG. 12A and FIG. 12B, and further that the pulse peaks are clearly seen even at 180 seconds and after, well into movement and/or exercise, in FIG. 12B.

In the calculations of this example, a simplistic but fast way to solve for the compartments was to consider venous blood to be 70% saturated, and for arterial blood to be exactly 100% saturated. Solving only for deoxygenated blood yields changes that must be only venous, as arterial blood has no venous blood in this simplistic analysis. Since venous blood is 30% oxygenated and 70% deoxygenated, the amount of total amount of venous blood changes can be calculated from the deoxyhemoglobin change plus an additional volume change of 30/70th of the deoxyhemoglobin change (that is an additional 30% volume that is oxygenated for every volume of venous blood that is deoxygenated). Removing the oxygenated component of the venous blood leaves a change in this example that must only be the arterial compartment change, which is far more pulse-driven than gravity- and body-position-driven. This allows a pulse to easily be seen, as shown in FIG. 12B.

Several important things are taught by the above example.

First, it is important to note that such a 70%/100% assumption is not required. Iterative methods can determine the ratios that best fit the data, or tissue oximetry and pulse oximetry can be used to measure these values more precisely, allowing accurate numbers to be used in the compartmental calculations.

Second, mathematical methods of solving such multiple equations are known. For example, one can apply multiple linear equations, where the values in the equation are: (1) an array of measured data within each waveband, (2) the corresponding absorbances, such as blood with and without oxygen, bilirubin, water, or fat, and (3) the result vector, which yields the concentrations (or changes in concentration) over time. In such an example, if the measured data is an N-element 1-D array named B, representing the data measured at N wavebands, and the known coefficients of effective reflection absorbance (absorbance and scattering) of each of M substances at each of the N wavebands are in a M by N 2-D array (a matrix of coefficients) named A, while the concentrations of each substance to be determined are in an M-element 1-D array of unknowns called X, then the values of X can be determined as (after regularization such that the math works, such as making N=M) then X equals the matrix operation: A⁻¹B. The values for the array of coefficients can be found in publications, or may be experimentally estimated. Alternatively, simple algebra can be used to reduce the complexity of the calculations to mere ratios in certain conditions, or weighted nodal partial-least-squares analysis can be used for even a more complex analysis. All of these fall under the present invention if used to correct for distance and motion in a loose-fit or non-contact physiological monitoring.

As another example, the concentration changes over time can be further partitioned into compartments by time (separation based on frequency, which is different for heart and respiratory variations, for example), or by saturation (the total changes in blood volume and saturation can be analyzed as changes in multiple compartments (such as partition into a venous component of 70% saturation versus an arterial compartment of 98% saturation).

As before, reduction of the noise by half (an improvement in signal to noise of “one bit”) may be sufficient. However, the combined improvement of both corrections yields an estimated reduction by more than 99%, or roughly 8 effective bits of signal to noise improvement.

Several additional comments are now included.

First, it should be understood that the compartment analysis (arterial bloodstream vs. venous bloodstream vs skin surface) and the component substance analysis (hemoglobin, fat, water, skin) can be performed simultaneously, and that they are performed sequentially here for the purposes of clarity of illustration. Further, the analysis can be processed in an iterative manner, which optimizes the separation based on different values of arterial and venous saturation, or upon different time constants for respiratory versus cardiac function.

Next, there are other methods that can be applied to this analysis. Time filtering, such as using a Fourier Transform to place the data into frequency-space from time-space, as is known in the art of data analysis, and can separate a regular heart rate from the pulse effects of respiration, as is shown in a later example.

All of these fall within the scope of the present invention if used in a multispectral or compartmental (or both) analysis to extract non-contact or loose-fit physiological parameters such as heart rate, respiratory rate, R-R heart beat interval, pulse oximetry, or tissue oximetry, cardiac function, bilirubin levels, sweat levels, hydration status, fat/water levels or ratios, cholesterol levels, or the like.

### SUMMARY

In summary, the improved sensors have multiple expected and unexpected advantages that can result from using broadband white LED illuminators (or broadband ambient light sources) and spectrally-resolved detectors in mobile devices, especially when combined with integrated processing power. In certain applications, such as fitness applications, this improvement may occur without undue space and size constraints, and all without degrading or with improvement in output stability. We show that improved sensors can be achieved by (a) using broadband light, from the room or from a white LED source, and (b) using a sensor with multiple narrowband spectral filters built into a portable board, such that the improved sensor can even be embedded into watches, bracelets, pendants, phones, and even clothes. Sensitivity to hemoglobin and other tissue components in various compartments allows for quantitative detection of gestures and physiology, and improves data quality during movement, allowing non-contact operation. Such improved sensors may permit a light source and detector to be embedded into nearly any mobile device, such as into a smartphone, bracelet, pendant, shoe, clothing, or watch.

We have discovered an improved calorie sensor and monitoring method for mobile, wearable, non-contact, and remote use. Various sensor implementations, such as a phone and a watch, have been constructed and tested, in which a solid-state broadband white LED, and one or more sensors having spectral filters designed to pass certain predetermined wavebands of light to produce (if needed in the absence of adequate ambient light or to replace ambient light) a continuous, broadband light from 400 nm to 700 nm, and a spectrally resolved detection. The resulting data is passed to a processor and memory having programs for execution by the processor to determine a measure of calories, such as calories expended, calories ingested, calorie balance, or rate of calories expended, in part based on a noninvasive measure of respiration, such as respiratory rate, respiratory effort, respiratory depth, or respiratory variability. In one example, variations in components of the bloodstream over time such as hemoglobin and water are determined based on the detected light, and the measure of respiration is then determined based on the in components of the bloodstream over time. In addition, the sensor is sensitive to other physiology (e.g., heart rate, hydration, jaundice, alcohol levels), as well as to type and state (e.g., finger, hand, live, dead), for analysis and initiating actions based on the resulting determinations. This device has been built and tested in several configurations in models, animals, and humans, and has immediate application to several important problems, both medical and industrial, and thus constitutes an important advance in the art.

We claim:

1. A method for estimating calories in a living subject, comprising the steps of:
(a) noninvasively detecting light, said detected light being at least in part backscattered from or transmitted through the subject;
(b) determining a measure of respiration, said measure determined at least in part based on a function of a concentration of components of the bloodstream over time determined by analysis of the detected light; and,
(c) generating an output that is a function of the estimated calories expended or consumed by the subject, said output based at least in part on the measure of respiration.

2. A method for estimating calories in a living subject, comprising the steps of:
(a) noninvasively detecting broadband light after an interaction with the subject, and further after separating or filtering said broadband light into different wavebands of light prior to detection;
(b) determining a measure of respiration, said measure based at least in part on a spectral analysis of the detected light over an interval of time; and,
(c) generating an output that is a function of the estimated calories expended or consumed by the subject, said output based at least in part on the measure of respiration.

3. A method for estimating calories in a living subject, comprising the steps of:
(a) noninvasively detecting broadband light arriving after interaction with the subject, and further after separating or filtering said broadband light into different wavebands prior to detection, and generating spectral data;
(b) analyzing the spectral data to computationally partition the data into more than one physiological compartment of different temporal or physiological characteristics and to computationally separate the detected light into more than one blood or tissue components;
(c) determining a measure of respiration, said measure based at least in part on the computational partitioning and the computational separation; and,
(d) generating an output that is a function of the estimated calories expended or consumed by the subject, said output based at least in part on the measure of respiration.

4. The method of claim 1, 2, or 3, wherein the detected light is broadband ambient light.

5. The method of claim 1, 2, or 3, wherein the detected light is broadband LED light.

6. The method of claim 1, 2, or 3, wherein the output is a measure selected from the list of measures including calories expended, calories consumed, calorie balance, and a rate of calorie expenditure over a period of time.

7. The method of claim 1, 2, or 3, wherein the output is determined using a function of one or more of an estimated water, fat, or protein content of the blood.

8. The method of claim 1, 2, or 3, wherein the output is measure of a condition of calories consumed being more than calories expended.

9. The method of claim 1, 2, or 3, wherein the output is measure of a condition of calories consumed being less than calories expended.

10. A device for estimating calories in a living subject, comprising:
(a) at least one sensor configured to noninvasively detect light being backscattered from or transmitted through the subject; and,
(b) a processor, and memory storing one or more programs for execution by the processor, the one or more programs including instructions for determining at least a measure of respiration, said measure of respiration determined at least in part based on a measure of components of the bloodstream over time determined using the detected light, and for generating an output that is a function of the calories expended or consumed by the subject, said output based at least in part on the measure of respiration.

11. A device for estimating calories in a living subject, comprising:
(a) one or more sensors configured to noninvasively detect broadband light after interaction with the subject, each of said sensors further comprising at least one narrowband spectral filter configured to produce one or more sensor regions, each sensor region configured to be sensitive to a predetermined waveband of light; and,
(b) a processor, and memory storing one or more programs for execution by the processor, the one or more programs including instructions for determining at least a measure of respiration, said measure determined at least in part on a spectral analysis of detected light over an interval of time, and for generating an output that is a function of the calories expended or consumed by the subject, said output based at least in part on the measure of respiration.

12. A device for estimating calories in a living subject, comprising:
(a) one or more sensors configured to noninvasively detect broadband light after interaction with the subject, each sensor further comprising a narrowband spectral filter configured to be sensitive to a predetermined waveband of light; and,
(b) a processor, and memory storing one or more programs for execution by the processor, the one or more programs including instructions for generating spectral data using the detected light, analyzing the spectral data to computationally partition the data into more than one physiological compartment of different temporal or physiological characteristics and to computationally separate the detected light into more than one blood or tissue components, determining at least a measure of respiration at least in part based on the computational partitioning and computational separation, and generating an output that is a function of the calories expended or consumed by the subject, said output based at least in part on the measure of respiration.

13. A device for estimating calories in a living subject, comprising:
(a) a solid-state broadband white LED illuminator configured to illuminate a target site on the subject with broadband light;
(b) three or more sensors configured to noninvasively detect light returning after interaction with the subject, each sensor further comprising a narrowband spectral filter configured to be sensitive to a predetermined waveband of light; and,
(c) a processor, and memory storing one or more programs for execution by the processor, the one or more programs including instructions for generating spectral data using the detected light, determining at least a measure of respiration at least in part based on a spectral analysis of the spectral data, and generating an output that is a function of the calories expended or consumed by the subject, said output based at least in part on the measure of respiration.

14. The device of claim 10, 11, 12, or 13 wherein the device is configured as part of a system selected from the list of systems including a mobile personal health monitor, a mobile phone, a wearable device, wearable clothing, wearable glasses, a wearable bracelet, wearable earphones, wearable contact lenses, a security system, and a occupancy sensor.

15. The device of claim 10, 11, 12, or 13, further comprising at least one spectral filter deposited directly on the sensor, wherein the sensitivity of the detector to different wavelength bands is achieved using said at least one filter.

16. The device of claim 15, wherein the at least one of the at least one spectral filters is configured to be a narrowband spectral filter.

17. The device of claim 10, 11, 12, or 13, wherein the sensor is configured to operate in a non-contact manner with the subject.
